SEQUENCE LISTING (91 sequences)

SEQ ID NO: 1 - protein, 25 aa, Artificial Sequence
  framework region 1 of PD-1 antibody
QVQLVESGGGLVQPGGSLNLSCVAS

SEQ ID NO: 2 - protein, 25 aa, Artificial Sequence
  FR1 of anti-PD-1 single-domain antibody
QVQLVESGGGLVRPGGSLRLSCAAS

SEQ ID NO: 3 - protein, 25 aa, Artificial Sequence
  FR1 of anti-PD-1 single-domain antibody
QVQLVESGGGLVQPGGSLRLSCAAS

SEQ ID NO: 4 - protein, 25 aa, Artificial Sequence
  FR1 of anti-PD-1 single-domain antibody
QVQLVESGGGLVQPGGSLRLSCAAS

SEQ ID NO: 5 - protein, 8 aa, Artificial Sequence
  CDR1 of anti-PD-1 single-domain antibody
GNVFIIDV

SEQ ID NO: 6 - protein, 8 aa, Artificial Sequence
  CDR1 of anti-PD-1 single-domain antibody
GSVFIIDV

SEQ ID NO: 7 - protein, 17 aa, Artificial Sequence
  FR2 of anti-PD-1 single-domain antibody
MAWYRQAPGKQRELVAQ

SEQ ID NO: 8 - protein, 17 aa, Artificial Sequence
  FR2 of anti-PD-1 single-domain antibody
MAWYRQAPGKGLELVAQ

SEQ ID NO: 9 - protein, 7 aa, Artificial Sequence
  CDR2 of anti-PD-1 single-domain antibody
IINGDAK

SEQ ID NO: 10 - protein, 7 aa, Artificial Sequence
  CDR2 of anti-PD-1 single-domain antibody
IINGGAT

SEQ ID NO: 11 - protein, 7 aa, Artificial Sequence
  CDR2 of anti-PD-1 single-domain antibody
IINGGAS

SEQ ID NO: 12 - protein, 7 aa, Artificial Sequence
  CDR2 of anti-PD-1 single-domain antibody
IISGDHA

SEQ ID NO: 13 - protein, 38 aa, Artificial Sequence
  FR3 of anti-PD-1 single-domain antibody
YYVDSVKGRFTISRDNTKTTLYLQMNSLKPDDTAIYYC

SEQ ID NO: 14 - protein, 38 aa, Artificial Sequence
  FR3 of anti-PD-1 single-domain antibody
YYADSVKGRFTISRDDTKTTLNLQMNSLKPDDTAVYYC

SEQ ID NO: 15 - protein, 38 aa, Artificial Sequence
  FR3 of anti-PD-1 single-domain antibody
YYADSVKGRFTISRDNTKTTLFLQMNSLKPADTAVYYC

SEQ ID NO: 16 - protein, 38 aa, Artificial Sequence
  FR3 of anti-PD-1 single-domain antibody
YYADSVKGRFTISRDNSKNTLYLQMNSLRAEDTAVYYC

SEQ ID NO: 17 - protein, 14 aa, Artificial Sequence
  CDR3 of anti-PD-1 single-domain antibody
SAEKFNPYRDSTNF

SEQ ID NO: 18 - protein, 14 aa, Artificial Sequence
  CDR3 of anti-PD-1 single-domain antibody
SAEKFNPYRDSTHF

SEQ ID NO: 19 - protein, 14 aa, Artificial Sequence
  CDR3 of anti-PD-1 single-domain antibody
NAEKFNPYRDSTNF

SEQ ID NO: 20 - protein, 14 aa, Artificial Sequence
  CDR3 of anti-PD-1 single-domain antibody
SAEKFNPYRDSTYF

SEQ ID NO: 21 - protein, 14 aa, Artificial Sequence
  CDR3 of anti-PD-1 single-domain antibody
SAEQFNPYRDSTYF

SEQ ID NO: 22 - protein, 348 aa, Artificial Sequence
  Anti-PD1-1A4-Fc
QVQLVESGGGLVQPGGSLNLSCVASGNVFIIDVMAWYRQAPGKQRELVAQIINGDAKYYVDSVKGRFTISRDNTKTTLYLQMNSLKPDDTAIYYCSAEKFNPYRDSTNFWGQGTQVTVSSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 23 - protein, 348 aa, Artificial Sequence
  Anti-PD1-1A9-Fc
QVQLVESGGGLVQPGGSLNLSCVASGNVFIIDVMAWYRQAPGKQRELVAQIINGGATYYADSVKGRFTISRDDTKTTLNLQMNSLKPDDTAVYYCSAEKFNPYRDSTHFWGQGTQVTVSAESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 24 - protein, 348 aa, Artificial Sequence
  Anti-PD1-1H1-Fc
QVQLVESGGGLVQPGGSLNLSCVASGSVFIIDVMAWYRQAPGKQRELVAQIINGGASYYADSVKGRFTISRDNTKTTLFLQMNSLKPADTAVYYCNAEKFNPYRDSTNFWGQGTQVTVSSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 25 - protein, 348 aa, Artificial Sequence
  Anti-PD1-1D3-Fc
QVQLVESGGGLVQPGGSLNLSCVASGSVFIIDVMAWYRQAPGKQRELVAQIINGGATYYADSVKGRFTISRDDTKTTLYLQMNSLKPDDTAVYYCSAEKFNPYRDSTHFWGQGTQVTVSSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 26 - protein, 348 aa, Artificial Sequence
  Anti-PD1-2C4-Fc
QVQLVESGGGLVRPGGSLRLSCAASGSVFIIDVMAWYRQAPGKQRELVAQIISGDHAYYADSVKGRFTISRDDTKTTLYLQMNSLKPDDTAVYYCSAEKFNPYRDSTYFWGQGTQVTVSSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 27 - protein, 348 aa, Artificial Sequence
  Anti-PD1-2D9-Fc
QVQLVESGGGLVQPGGSLRLSCAASGSVFIIDVMAWYRQAPGKQRELVAQIINGDAKYYADSVKGRFTISRDNTKTTLYLQMNSLKPDDTAVYYCSAEQFNPYRDSTYFWGQGTQVTVSSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 28 - protein, 38 aa, Artificial Sequence
  FR3 of anti-PD-1 single-domain antibody
YYADSVKGRFTISRDDTKTTLYLQMNSLKPDDTAVYYC

SEQ ID NO: 29 - protein, 38 aa, Artificial Sequence
  FR3 of anti-PD-1 single-domain antibody
YYADSVKGRFTISRDNTKTTLYLQMNSLKPDDTAVYYC

SEQ ID NO: 30 - protein, 737 aa, Artificial Sequence
  Monkey RhPD1-HSA fusion protein
LESPDRPWNAPTFSPALLLVTEGDNATFTCSFSNASESFVLNWYRMSPSNQTDKLAAFPEDRSQPGQDCRFRVTRLPNGRDFHMSVVRARRNDSGTYLCGAISLAPKAQIKESLRAELRVTERRAEVPTAHPSPSPRPAGQFQGGGGSGGGGDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 31 - protein, 11 aa, Artificial Sequence
  FR4 of anti-PD-1 single-domain antibody
WGQGTQVTVSS

SEQ ID NO: 32 - protein, 11 aa, Artificial Sequence
  FR4 of anti-PD-1 single-domain antibody
WGQGTQVTVSA

SEQ ID NO: 33 - protein, 11 aa, Artificial Sequence
  FR4 of anti-PD-1 single-domain antibody
WGQGTLVTVSS

SEQ ID NO: 34 - protein, 120 aa, Artificial Sequence
  anti-PD-1 single-domain antibody
QVQLVESGGGLVQPGGSLNLSCVASGNVFIIDVMAWYRQAPGKQRELVAQIINGDAKYYVDSVKGRFTISRDNTKTTLYLQMNSLKPDDTAIYYCSAEKFNPYRDSTNFWGQGTQVTVSS

SEQ ID NO: 35 - protein, 120 aa, Artificial Sequence
  anti-PD-1 single-domain antibody
QVQLVESGGGLVQPGGSLNLSCVASGNVFIIDVMAWYRQAPGKQRELVAQIINGGATYYADSVKGRFTISRDDTKTTLNLQMNSLKPDDTAVYYCSAEKFNPYRDSTHFWGQGTQVTVSA

SEQ ID NO: 36 - protein, 120 aa, Artificial Sequence
  anti-PD-1 single-domain antibody
QVQLVESGGGLVQPGGSLNLSCVASGSVFIIDVMAWYRQAPGKQRELVAQIINGGASYYADSVKGRFTISRDNTKTTLFLQMNSLKPADTAVYYCNAEKFNPYRDSTNFWGQGTQVTVSS

SEQ ID NO: 37 - protein, 120 aa, Artificial Sequence
  anti-PD-1 single-domain antibody
QVQLVESGGGLVQPGGSLNLSCVASGSVFIIDVMAWYRQAPGKQRELVAQIINGGATYYADSVKGRFTISRDDTKTTLYLQMNSLKPDDTAVYYCSAEKFNPYRDSTHFWGQGTQVTVSS

SEQ ID NO: 38 - protein, 120 aa, Artificial Sequence
  anti-PD-1 single-domain antibody
QVQLVESGGGLVRPGGSLRLSCAASGSVFIIDVMAWYRQAPGKQRELVAQIISGDHAYYADSVKGRFTISRDDTKTTLYLQMNSLKPDDTAVYYCSAEKFNPYRDSTYFWGQGTQVTVSS

SEQ ID NO: 39 - protein, 120 aa, Artificial Sequence
  anti-PD-1 single-domain antibody
QVQLVESGGGLVQPGGSLRLSCAASGSVFIIDVMAWYRQAPGKQRELVAQIINGDAKYYADSVKGRFTISRDNTKTTLYLQMNSLKPDDTAVYYCSAEQFNPYRDSTYFWGQGTQVTVSS

SEQ ID NO: 40 - protein, 120 aa, Artificial Sequence
  anti-PD-1 single-domain antibody
QVQLVESGGGLVQPGGSLRLSCAASGNVFIIDVMAWYRQAPGKQRELVAQIINGDAKYYADSVKGRFTISRDNSKNTLYLQMNSLRAEDTAVYYCSAEKFNPYRDSTNFWGQGTLVTVSS

SEQ ID NO: 41 - protein, 120 aa, Artificial Sequence
  anti-PD-1 single-domain antibody
QVQLVESGGGLVQPGGSLRLSCAASGNVFIIDVMAWYRQAPGKQRELVAQIINGGATYYADSVKGRFTISRDNSKNTLYLQMNSLRAEDTAVYYCSAEKFNPYRDSTHFWGQGTLVTVSS

SEQ ID NO: 42 - protein, 120 aa, Artificial Sequence
  anti-PD-1 single-domain antibody
QVQLVESGGGLVQPGGSLRLSCAASGSVFIIDVMAWYRQAPGKQRELVAQIINGGASYYADSVKGRFTISRDNSKNTLYLQMNSLRAEDTAVYYCNAEKFNPYRDSTNFWGQGTLVTVSS

SEQ ID NO: 43 - protein, 120 aa, Artificial Sequence
  anti-PD-1 single-domain antibody
QVQLVESGGGLVQPGGSLRLSCAASGSVFIIDVMAWYRQAPGKQRELVAQIINGGATYYADSVKGRFTISRDNSKNTLYLQMNSLRAEDTAVYYCSAEKFNPYRDSTHFWGQGTLVTVSS

SEQ ID NO: 44 - protein, 120 aa, Artificial Sequence
  anti-PD-1 single-domain antibody
QVQLVESGGGLVQPGGSLRLSCAASGSVFIIDVMAWYRQAPGKQRELVAQIISGDHAYYADSVKGRFTISRDNSKNTLYLQMNSLRAEDTAVYYCSAEKFNPYRDSTYFWGQGTLVTVSS

SEQ ID NO: 45 - protein, 120 aa, Artificial Sequence
  anti-PD-1 single-domain antibody
QVQLVESGGGLVQPGGSLRLSCAASGSVFIIDVMAWYRQAPGKQRELVAQIINGDAKYYADSVKGRFTISRDNSKNTLYLQMNSLRAEDTAVYYCSAEQFNPYRDSTYFWGQGTLVTVSS

SEQ ID NO: 46 - protein, 120 aa, Artificial Sequence
  anti-PD-1 single-domain antibody
QVQLVESGGGLVQPGGSLRLSCAASGNVFIIDVMAWYRQAPGKGLELVAQIINGDAKYYADSVKGRFTISRDNSKNTLYLQMNSLRAEDTAVYYCSAEKFNPYRDSTNFWGQGTLVTVSS

SEQ ID NO: 47 - protein, 120 aa, Artificial Sequence
  anti-PD-1 single-domain antibody
QVQLVESGGGLVQPGGSLRLSCAASGNVFIIDVMAWYRQAPGKGLELVAQIINGGATYYADSVKGRFTISRDNSKNTLYLQMNSLRAEDTAVYYCSAEKFNPYRDSTHFWGQGTLVTVSS

SEQ ID NO: 48 - protein, 120 aa, Artificial Sequence
  anti-PD-1 single-domain antibody
QVQLVESGGGLVQPGGSLRLSCAASGSVFIIDVMAWYRQAPGKGLELVAQIINGGASYYADSVKGRFTISRDNSKNTLYLQMNSLRAEDTAVYYCNAEKFNPYRDSTNFWGQGTLVTVSS

SEQ ID NO: 49 - protein, 120 aa, Artificial Sequence
  anti-PD-1 single-domain antibody
QVQLVESGGGLVQPGGSLRLSCAASGSVFIIDVMAWYRQAPGKGLELVAQIINGGATYYADSVKGRFTISRDNSKNTLYLQMNSLRAEDTAVYYCSAEKFNPYRDSTHFWGQGTLVTVSS

SEQ ID NO: 50 - protein, 120 aa, Artificial Sequence
  anti-PD-1 single-domain antibody
QVQLVESGGGLVQPGGSLRLSCAASGSVFIIDVMAWYRQAPGKGLELVAQIISGDHAYYADSVKGRFTISRDNSKNTLYLQMNSLRAEDTAVYYCSAEKFNPYRDSTYFWGQGTLVTVSS

SEQ ID NO: 51 - protein, 120 aa, Artificial Sequence
  anti-PD-1 single-domain antibody
QVQLVESGGGLVQPGGSLRLSCAASGSVFIIDVMAWYRQAPGKGLELVAQIINGDAKYYADSVKGRFTISRDNSKNTLYLQMNSLRAEDTAVYYCSAEQFNPYRDSTYFWGQGTLVTVSS

SEQ ID NO: 52 - protein, 231 aa, Artificial Sequence
  immunoglobulin Fc region of anti-PD-1 single-domain antibody
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 53 - protein, 228 aa, Artificial Sequence
  immunoglobulin Fc region of anti-PD-1 single-domain antibody
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 54 - protein, 737 aa, Artificial Sequence
  fusion protein hPD1-HSA
LDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQGGGGSGGGGDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 55 - protein, 459 aa, Artificial Sequence
  bio-hPDL1-FC
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTDIEGRMDPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 56 - protein, 381 aa, Artificial Sequence
  fusion protein hPD1-Fc
LDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQDIEGRMDPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 57 - protein, 739 aa, Artificial Sequence
  mouse mPD1-HSA
LEVPNGPWRSLTFYPAWLTVSEGANATFTCSLSNWSEDLMLNWNRLSPSNQTEKQAAFCNGLSQPVQDARFQIIQLPNRHDFHMNILDTRRNDSGIYLCGAISLHPKAKIEESPGAELVVTERILETSTRYPSPSPKPEGRFQGMGGGGSGGGGDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 58 - protein, 348 aa, Artificial Sequence
  fusion protein
QVQLVESGGGLVQPGGSLRLSCAASGNVFIIDVMAWYRQAPGKQRELVAQIINGDAKYYADSVKGRFTISRDNSKNTLYLQMNSLRAEDTAVYYCSAEKFNPYRDSTNFWGQGTLVTVSSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 59 - protein, 348 aa, Artificial Sequence
  fusion protein
QVQLVESGGGLVQPGGSLRLSCAASGNVFIIDVMAWYRQAPGKQRELVAQIINGGATYYADSVKGRFTISRDNSKNTLYLQMNSLRAEDTAVYYCSAEKFNPYRDSTHFWGQGTLVTVSSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 60 - protein, 358 aa, Artificial Sequence
  fusion protein
QVQLVESGGGLVQPGGSLRLSCAASGSVFIIDVMAWYRQAPGKQRELVAQIINGGASYYADSVKGRFTISRDNSKNTLYLQMNSLRAEDTAVYYCNAEKFNPYRDSTNFWGQGTLVTVSSGGGGSGGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 61 - protein, 348 aa, Artificial Sequence
  fusion protein
QVQLVESGGGLVQPGGSLRLSCAASGSVFIIDVMAWYRQAPGKQRELVAQIINGGATYYADSVKGRFTISRDNSKNTLYLQMNSLRAEDTAVYYCSAEKFNPYRDSTHFWGQGTLVTVSSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 62 - protein, 348 aa, Artificial Sequence
  fusion protein
QVQLVESGGGLVQPGGSLRLSCAASGSVFIIDVMAWYRQAPGKQRELVAQIISGDHAYYADSVKGRFTISRDNSKNTLYLQMNSLRAEDTAVYYCSAEKFNPYRDSTYFWGQGTLVTVSSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 63 - protein, 348 aa, Artificial Sequence
  fusion protein
QVQLVESGGGLVQPGGSLRLSCAASGSVFIIDVMAWYRQAPGKQRELVAQIINGDAKYYADSVKGRFTISRDNSKNTLYLQMNSLRAEDTAVYYCSAEQFNPYRDSTYFWGQGTLVTVSSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 64 - protein, 348 aa, Artificial Sequence
  fusion protein
QVQLVESGGGLVQPGGSLRLSCAASGNVFIIDVMAWYRQAPGKGLELVAQIINGDAKYYADSVKGRFTISRDNSKNTLYLQMNSLRAEDTAVYYCSAEKFNPYRDSTNFWGQGTLVTVSSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 65 - protein, 348 aa, Artificial Sequence
  fusion protein
QVQLVESGGGLVQPGGSLRLSCAASGNVFIIDVMAWYRQAPGKGLELVAQIINGGATYYADSVKGRFTISRDNSKNTLYLQMNSLRAEDTAVYYCSAEKFNPYRDSTHFWGQGTLVTVSSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 66 - protein, 348 aa, Artificial Sequence
  fusion protein
QVQLVESGGGLVQPGGSLRLSCAASGSVFIIDVMAWYRQAPGKGLELVAQIINGGASYYADSVKGRFTISRDNSKNTLYLQMNSLRAEDTAVYYCNAEKFNPYRDSTNFWGQGTLVTVSSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 67 - protein, 348 aa, Artificial Sequence
  fusion protein
QVQLVESGGGLVQPGGSLRLSCAASGSVFIIDVMAWYRQAPGKGLELVAQIINGGATYYADSVKGRFTISRDNSKNTLYLQMNSLRAEDTAVYYCSAEKFNPYRDSTHFWGQGTLVTVSSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 68 - protein, 348 aa, Artificial Sequence
  fusion protein
QVQLVESGGGLVQPGGSLRLSCAASGSVFIIDVMAWYRQAPGKGLELVAQIISGDHAYYADSVKGRFTISRDNSKNTLYLQMNSLRAEDTAVYYCSAEKFNPYRDSTYFWGQGTLVTVSSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 69 - protein, 348 aa, Artificial Sequence
  fusion protein
QVQLVESGGGLVQPGGSLRLSCAASGSVFIIDVMAWYRQAPGKGLELVAQIINGDAKYYADSVKGRFTISRDNSKNTLYLQMNSLRAEDTAVYYCSAEQFNPYRDSTYFWGQGTLVTVSSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 70 - protein, 23 aa, Artificial Sequence
  Control 1
AAAVLLDGSFSLLAPLVPYKYDY

SEQ ID NO: 71 - protein, 18 aa, Artificial Sequence
  Control 2
NAEAATWGGSSWYGPYEY

SEQ ID NO: 72 - protein, 19 aa, Artificial Sequence
  Control 3
ARDERVYSDIDFFRPFDYG

SEQ ID NO: 73 - protein, 116 aa, Artificial Sequence
  humanized antibody DP-47
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKSYGAFDYWGQGTLVTVSS

SEQ ID NO: 74 - protein, 231 aa, Artificial Sequence
  immunoglobulin Fc region of anti-PD-1 single-domain antibody
EPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 75 - protein, 231 aa, Artificial Sequence
  immunoglobulin Fc region of anti-PD-1 single-domain antibody
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNWALPAPISKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 76 - protein, 231 aa, Artificial Sequence
  immunoglobulin Fc region of anti-PD-1 single-domain antibody
EPKSSDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 77 - protein, 231 aa, Artificial Sequence
  immunoglobulin Fc region of anti-PD-1 single-domain antibody
EPKSSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 78 - protein, 231 aa, Artificial Sequence
  immunoglobulin Fc region of anti-PD-1 single-domain antibody
EPKSSDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 79 - protein, 228 aa, Artificial Sequence
  immunoglobulin Fc region of anti-PD-1 single-domain antibody
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 80 - DNA, 360 bp, Artificial Sequence
  hu1A4V1
caggtgcagctcgtggagtctgggggaggcttggtgcagcctggggggtctctgagactctcctgtgcagcctctggaaatgtcttcattatcgatgtcatggcctggtaccgccaggctccagggaagcagcgcgaattggtcgcgcaaatcatcaatggtgatgcgaaatactatgcagactccgtgaagggtcgattcaccatctccagagacaacagcaagaacacgctgtatctgcaaatgaacagcctgcgggctgaggacacggccgtctattactgtagtgcagagaaatttaacccctatcgggatagtacgaatttctggggccaggggaccctggtcaccgtgtcttct

SEQ ID NO: 81 - DNA, 360 bp, Artificial Sequence
  hu1A9V1
caggtgcagctcgtggagtctgggggaggcttggtgcagcctggggggtctctgagactctcctgtgcagcctctggaaatgtcttcattatcgatgtcatggcctggtaccgccaggctccagggaagcagcgcgaattggtcgcgcaaatcatcaatggtggtgcgacatactatgcagactccgtgaagggtcgattcaccatctccagagacaacagcaagaacacgctgtatctgcaaatgaacagcctgcgggctgaggacacggccgtctattactgtagtgcagagaaatttaacccctatcgggatagtacgcatttctggggccaggggaccctggtcaccgtgtcttct

SEQ ID NO: 82 - DNA, 360 bp, Artificial Sequence
  hu1H1V1
caggtgcagctcgtggagtctgggggaggcttggtgcagcctggggggtctctgagactctcctgtgcagcctctggaagtgtcttcattatcgatgtcatggcctggtaccgccaggctccagggaagcagcgcgaattggtcgcgcaaatcatcaatggtggtgcgagttactatgcagactccgtgaagggtcgattcaccatctccagagacaacagcaagaacacgctgtatctgcaaatgaacagcctgcgggctgaggacacggccgtctattactgtaatgcagagaaatttaacccctatcgggatagtacgaatttctggggccaggggaccctggtcaccgtgtcttct

SEQ ID NO: 83 - DNA, 360 bp, Artificial Sequence
  hu1D3V1
caggtgcagctcgtggagtctgggggaggcttggtgcagcctggggggtctctgagactctcctgtgcagcctctggaagtgtcttcattatcgatgtcatggcctggtaccgccaggctccagggaagcagcgcgaattggtcgcgcaaatcatcaatggtggtgcgacatactatgcagactccgtgaagggtcgattcaccatctccagagacaacagcaagaacacgctgtatctgcaaatgaacagcctgcgggctgaggacacggccgtctattactgtagtgcagagaaatttaacccctatcgggatagtacgcatttctggggccaggggaccctggtcaccgtgtcttct

SEQ ID NO: 84 - DNA, 360 bp, Artificial Sequence
  hu2C4V1
caggtgcagctcgtggagtctgggggaggcttggtgcagcctggggggtctctgagactctcctgtgcagcctctggaagtgtcttcattatcgatgtcatggcctggtaccgccaggctccagggaagcagcgcgaattggtcgcgcaaatcatcagtggtgatcacgcatactatgcagactccgtgaagggtcgattcaccatctccagagacaacagcaagaacacgctgtatctgcaaatgaacagcctgcgggctgaggacacggccgtctattactgtagtgcagagaaatttaacccctatcgggatagtacgtatttctggggccaggggaccctggtcaccgtgtcttct

SEQ ID NO: 85 - DNA, 360 bp, Artificial Sequence
  hu2D9V1
caggtgcagctcgtggagtctgggggaggcttggtgcagcctggggggtctctgagactctcctgtgcagcctctggaagtgtcttcattatcgatgtcatggcctggtaccgccaggctccagggaagcagcgcgaattggtcgcgcaaatcatcaatggtgatgcgaaatactatgcagactccgtgaagggtcgattcaccatctccagagacaacagcaagaacacgctgtatctgcaaatgaacagcctgcgggctgaggacacggccgtctattactgtagtgcagagcaatttaacccctatcgggatagtacgtatttctggggccaggggaccctggtcaccgtgtcttct

SEQ ID NO: 86 - DNA, 360 bp, Artificial Sequence
  hu1A4V3
caggtgcagctcgtggagtctgggggaggcttggtgcagcctggggggtctctgagactctcctgtgcagcctctggaaatgtcttcattatcgatgtcatggcctggtaccgccaagctccagggaagggcctggagctggtcgcacaaatcatcaatggtgatgcgaaatactatgcagactccgtgaagggtcgattcaccatctccagagacaacagcaagaacacgctgtatctgcaaatgaacagcctgcgggctgaggacacggccgtctattactgtagtgcagagaaatttaacccctatcgggatagtacgaatttctggggccaggggaccctggtcaccgtgtcttct

SEQ ID NO: 87 - DNA, 360 bp, Artificial Sequence
  hu1A9V3
caggtgcagctcgtggagtctgggggaggcttggtgcagcctggggggtctctgagactctcctgtgcagcctctggaaatgtcttcattatcgatgtcatggcctggtaccgccaagctccagggaagggcctggagctggtcgcacaaatcatcaatggtggtgcgacatactatgcagactccgtgaagggtcgattcaccatctccagagacaacagcaagaacacgctgtatctgcaaatgaacagcctgcgggctgaggacacggccgtctattactgtagtgcagagaaatttaacccctatcgggatagtacgcatttctggggccaggggaccctggtcaccgtgtcttct

SEQ ID NO: 88 - DNA, 360 bp, Artificial Sequence
  hu1H1V3
caggtgcagctcgtggagtctgggggaggcttggtgcagcctggggggtctctgagactctcctgtgcagcctctggaagtgtcttcattatcgatgtcatggcctggtaccgccaagctccagggaagggcctggagctggtcgcacaaatcatcaatggtggtgcgagttactatgcagactccgtgaagggtcgattcaccatctccagagacaacagcaagaacacgctgtatctgcaaatgaacagcctgcgggctgaggacacggccgtctattactgtaatgcagagaaatttaacccctatcgggatagtacgaatttctggggccaggggaccctggtcaccgtgtcttct

SEQ ID NO: 89 - DNA, 360 bp, Artificial Sequence
  hu1D3V3
caggtgcagctcgtggagtctgggggaggcttggtgcagcctggggggtctctgagactctcctgtgcagcctctggaagtgtcttcattatcgatgtcatggcctggtaccgccaagctccagggaagggcctggagctggtcgcacaaatcatcaatggtggtgcgacatactatgcagactccgtgaagggtcgattcaccatctccagagacaacagcaagaacacgctgtatctgcaaatgaacagcctgcgggctgaggacacggccgtctattactgtagtgcagagaaatttaacccctatcgggatagtacgcatttctggggccaggggaccctggtcaccgtgtcttct

SEQ ID NO: 90 - DNA, 360 bp, Artificial Sequence
  hu2C4V3
caggtgcagctcgtggagtctgggggaggcttggtgcagcctggggggtctctgagactctcctgtgcagcctctggaagtgtcttcattatcgatgtcatggcctggtaccgccaagctccagggaagggcctggagctggtcgcacaaatcatcagtggtgatcacgcatactatgcagactccgtgaagggtcgattcaccatctccagagacaacagcaagaacacgctgtatctgcaaatgaacagcctgcgggctgaggacacggccgtctattactgtagtgcagagaaatttaacccctatcgggatagtacgtatttctggggccaggggaccctggtcaccgtgtcttct

SEQ ID NO: 91 - DNA, 360 bp, Artificial Sequence
  hu2D9V3
caggtgcagctcgtggagtctgggggaggcttggtgcagcctggggggtctctgagactctcctgtgcagcctctggaagtgtcttcattatcgatgtcatggcctggtaccgccaagctccagggaagggcctggagctggtcgcacaaatcatcaatggtgatgcgaaatactatgcagactccgtgaagggtcgattcaccatctccagagacaacagcaagaacacgctgtatctgcaaatgaacagcctgcgggctgaggacacggccgtctattactgtagtgcagagcaatttaacccctatcgggatagtacgtatttctggggccaggggaccctggtcaccgtgtcttct

(19) United States
(12) Patent Application Publication
(10) Pub. No.: US 2024/0092908 A1
(43) Pub. Date: Mar. 21, 2024

(54) ANTI-PD-1 SINGLE-DOMAIN ANTIBODY

(71) Applicant: ZHEJIANG DOER BIOLOGICS CO., LTD., Hangzhou (CN)

(72) Inventor: Yanshan HUANG, Hangzhou (CN)

(73) Assignee: ZHEJIANG DOER BIOLOGICS CO., LTD., Hangzhou (CN)

(21) Appl. No.: 18/270,225

(22) PCT Filed: Dec. 31, 2020
(86) PCT No.: PCT/CN2020/142053

§ 371 Date: Jun. 29, 2023

(51) Int. Cl. C07K 16/28; A61P 35/00

(52) U.S. Cl. CPC C07K 16/2818; A61P 35/00; C07K 2317/14; C07K 2317/24; C07K 2317/52; C07K 2317/565; C07K 2317/567; C07K 2317/569

(57) ABSTRACT

The present application generally relates to the field of biotechnology and, in particular, to an anti-PD-1 single-domain antibody. The present application provides an anti-PD-1 single-domain antibody, which includes complementary determining regions including CDR1 having an amino acid sequence as set forth in one of SEQ ID Nos. 5 to 6, CDR2 having an amino acid sequence as set forth in one of SEQ ID Nos. 9 to 12, and CDR3 having an amino acid sequence as set forth in one of SEQ ID Nos. 17 to 21. The anti-PD-1 single-domain antibody provided by the present disclosure has specific binding ability for PD-1 and can be used to further construct fusion proteins, etc., which can also increase IFN-γ and/or IL-2 expression in T lymphocytes and can effectively inhibit tumor growth, and therefore has good prospects for industrialization.

### FIELD OF THE INVENTION

The present disclosure generally relates to the field of biotechnology and, in particular, to an anti-PD-1 single-domain antibody.

### BACKGROUND OF THE INVENTION

Programmed death receptor-1 (PD-1) is an important immune inhibitory molecule that, when bound to its ligand, inhibits the activation of T cells. PD-1 is widely expressed mainly on surfaces of activated CD4+ and CD8+ T cells, natural killer cells (NK), macrophages, B cells, and some tumor cells.

The ligands of PD-1 include programmed death ligand 1 (PD-L1) and programmed death ligand 2 (PD-L2), of which PD-L1 is the main ligand.

During tumor development, cancer cells induce apoptosis of T cells by upregulating PD-L1 expression to avoid clearance of the cancer cells by the immune system, which leads to disease progression. In recent years, monoclonal antibody drugs targeting PD-1/PD-L1 proteins have been used to kill tumor cells by blocking the binding of PD-1/PD-L1, which in turn promotes the activation and proliferation of T cells in vivo, and has been applied in the treatment of various tumor-related diseases, such as melanoma, lymphoma, bladder cancer, non-small cell lung cancer, head and neck cancer, and colon cancer, with remarkable efficacy. Therefore, the PD1/PD-L1 pathway has become an important focal point for anti-tumor drug research. Currently, antibody drugs that inhibit the PD1/PD-L1 pathway have achieved great clinical success, among which, Nivolumab from Bristol-Myers Squibb, Pembrolizumab from Merck Sharp & Dohme, Atezolizumab from Roche, Avelumab from Merck, Durvalumab from AstraZeneca and Regeneron's Cemiplimab are available on the market. Antibody drugs that inhibit the PD1/PD-L1 pathway have become the most promising segment of the oncology therapeutic market.

Compared to macromolecular monoclonal antibodies, single-domain antibodies (aka VHH), especially those derived from alpacas, are gradually becoming a rising star in the field of tumor treatment. This is due to the unique properties of alpaca-derived single-domain antibodies: 1) they have high homology with human VH families 3 and 4, making them weakly immunogenic; 2) they have a small molecular weight of only about 15 kDa, and a simple structure, making it easy for them to express in large quantities in microorganisms and easy to purify. The unique properties and low cost of single-domain antibodies have greatly expanded their scope of applications, manifesting their value in the treatment and diagnosis of diseases. However, with current technology, obtaining high-affinity, high-specificity single-domain antibodies with industrialization prospects is still not an easy task. This is because screening antibodies that bind to antigens using only three complementarity-determining regions (CDRs) is much more difficult than screening antibodies that bind to antigens using six CDRs, which is also why the mainstream antibodies currently being commercialized or in preclinical research are still high molecular weight monoclonal antibodies.

### SUMMARY OF THE INVENTION

The present disclosure provides an anti-PD-1 single-domain antibody.

A first aspect of the present disclosure provides an anti-PD-1 single-domain antibody, which includes complementary determining regions including CDR1 having an amino acid sequence as set forth in one of SEQ ID Nos. 5 to 6, CDR2 having an amino acid sequence as set forth in one of SEQ ID Nos. 9 to 12, and CDR3 having an amino acid sequence as set forth in one of SEQ ID Nos. 17 to 21.

Another aspect of the present disclosure provides a fusion protein, including a first domain and a second domain, wherein the first domain includes the anti-PD-1 single-domain antibody, and the second domain includes a domain capable of prolonging the in vivo half-life of the fusion protein and/or a domain capable of binding to effector cells.

Another aspect of the present disclosure provides an isolated polynucleotide encoding the anti-PD-1 single-domain antibody, or the fusion protein.

Another aspect of the present disclosure provides a construct including the isolated polynucleotide.

Another aspect of the present disclosure provides an expression system for an antibody, which includes the construct or incorporates the exogenous polynucleotide into the genome.

Another aspect of the present disclosure provides a method for preparing the anti-PD-1 single-domain antibody, or the fusion protein, wherein the method includes: culturing the expression system for the antibody under conditions suitable for expressing the antibody or the fusion protein, thereby expressing the antibody or the fusion protein; and purifying and isolating the antibody or the fusion protein.

Another aspect of the present disclosure provides use of the anti-PD-1 single-domain antibody or the fusion protein in the preparation of a drug for the treatment of tumors.

Another aspect of the present disclosure provides a pharmaceutical composition including the anti-PD-1 single-domain antibody, or the fusion protein.

### BRIEF DESCRIPTION OF THE DRAWINGS

FIG. 1 is a schematic diagram showing blocking of PD-L1/PD-1 interaction by an Anti-PD1-Fc fusion protein in embodiment 6 of the present disclosure.

FIG. 2 is a schematic diagram showing in vitro cellular activity of an Anti-PD1-Fc fusion protein detected by a reporter gene method in embodiment 7 of the present disclosure.

FIG. 3 is a schematic diagram showing the effect of an Anti-PD1-Fc fusion protein on IL-2 secretion by mixed lymphocytes in embodiment 8 of the present disclosure.

FIG. 4 is a schematic diagram showing an inhibitory effect of an Anti-PD1-Fc fusion protein on cancer growth in embodiment 9 of the present disclosure.

### DETAILED DESCRIPTION

In order to make the inventive purpose, technical solutions, and beneficial technical effects of the present disclosure clearer, the present disclosure is described in further detail below in conjunction with exemplary embodiments, and those familiar with the field of technology can easily understand other advantages and efficacy of the present disclosure as disclosed herein.

Inventors of the present disclosure, after extensive exploratory research, accidentally discovered an anti-PD-1 single-domain antibody, which has good specificity and can effectively and specifically block PD-L1/PD-1 interaction, on the basis of which the present disclosure was completed.

In the present disclosure, the terms “programmed death receptor 1”, “PD-receptor 1”, “PD-1”, “PD1”, “CD279” can be used interchangeably, and may also refer to its variants, isoforms, homologs of different species, etc.

In the present disclosure, the term “monoclonal antibody” refers to a preparation of antibody molecules composed of a single type of molecule. Monoclonal antibodies display single binding specificity and affinity for specific epitopes.

In the present disclosure, the term “domain” (of a polypeptide or protein) generally refers to a folded protein structure that is capable of maintaining its tertiary structure independently of the rest of the protein. In general, domains are responsible for individual functional properties of a protein, and in most cases the addition or removal of a particular domain does not affect functions of the rest of the polypeptide or protein and/or other domains.

In the present disclosure, the term “single-(structural)-domain antibody (VHH)” generally refers to an immunoglobulin domain essentially including four “frame regions” which are referred to as “frame region 1” or “FR1”, “frame region 2” or “FR2”, “frame region 3” or “FR3”, and “frame region 4” or “FR4” in the art and hereinafter. The frame regions are separated by three “complementary determining regions” or “CDRs” which are referred to as “complementary determining region 1” or “CDR1”, “complementary determining region 2” or “CDR2”, and “complementary determining region 3” or “CDR3” in the art and hereinafter. Therefore, the general structure or sequence of a single domain antibody (VHH) can be expressed as follows: FR1-CDR1-FR2-CDR2-FR3-CDR3-FR4. Single domain antibodies (VHH) have antigen-binding sites that endow the antibody with specificity for antigens.

In the present disclosure, the terms “single structural domain antibody”, “single domain antibody”, “heavy chain single domain antibody”, “VHH domain”, “VHH”, “VHH antibody fragment”, and “VHH antibody” can be used interchangeably.

In the present disclosure, the term “IMGT numbering system” is refers to an integrated information system specifically for immunoglobulin (IG), T cell receptor (TCR) and major histocompatibility complex (MHC) of humans and other vertebrates, namely THE INTERNATIONAL IMMUNOGENETICS INFORMATION SYSTEM® (Lafranc et al., 2003, Dev. Comp. Immunol. 27(1):55-77). Log in to IMGT (http://www.imgt.org/IMGT_vquest) to analyze light chain and heavy chain genes of an antibody to determine the framework regions (FR) and complementarity determining regions (CDR) of the variable region. The “positions” of CDRs in the structure of the immunoglobulin variable domain are conserved among species and exists in a structure called a loop. Therefore, CDRs and frame residues can be easily identified by using the numbering system that aligns sequences of variable domains according to structural characteristics. This information can be used to transplant and replace a whole CDR of a immunoglobulin of one species into the frame of an acceptor normally derived from human antibodies. Unless otherwise specified, in the specification, claims and drawings of the present disclosure, anti-PD-1 single-domain antibodies are numbered following the IMGT numbering method to determine the CDRs and FRs.

In the present disclosure, the term “humanized antibody” refers to a molecule having antigen binding sites substantially derived from an immunoglobulin of a non-human species, and the remaining immunoglobulin structure of the molecule is based on the structure and/or sequence of a human immunoglobulin. The antigen binding sites may include complete variable domains fused to constant domains, or only complementary determining regions (CDRs) grafted into appropriate frame regions in the variable domains. The antigen binding sites may be wild-type, or modified by one or more amino acid substitutions, for example, modified to be more similar to human immunoglobulins. Some forms of humanized antibodies retain the full original CDR sequences (e.g., humanized single-domain antibodies containing all three CDRs from alpacas). Other forms have one or more CDRs that have been altered relative to the original antibodies.

In the present disclosure, “sequence identity” between two polypeptide sequences generally indicates the percentage of identical amino acids between the sequences. “Sequence similarity” indicates the percentage of amino acids that are identical or represent conservative amino acid substitutions. Methods for evaluating the degree of sequence identity between amino acids or nucleotides are known to those skilled in the art. For example, amino acid sequence identity is typically measured using sequence analysis software. For example, the BLAST program from the NCBI database may be used to determine identity.

For the purposes of the present disclosure, an “effective amount” of an agent generally refers to an amount necessary to induce physiological changes in the cell or tissue to which it is administered. A “therapeutically effective amount” of an agent (e.g., a pharmaceutical composition) is an amount that is effective in achieving a desired therapeutic or preventive outcome at the required dose and over the required time period. For example, eliminating, reducing, delaying, minimizing, or preventing the adverse effects of a disease.

In the present disclosure, an “individual” or a “subject” is usually a mammal. Mammals may be domesticated animals (e.g., cows, sheep, cats, dogs, horses, etc.), primates (e.g., human and non-human primates, e.g., monkeys, etc.), rabbits, and rodents (e.g., mice and rats), etc.

In the present disclosure, the term “pharmaceutical composition” refers to a formulation in a form such that the biological activity of the active ingredient contained therein is effective and does not contain other ingredients with unacceptable toxicities to a subject who would receive administration of the composition.

In the present disclosure, “pharmaceutically acceptable carriers” means components of the pharmaceutical compositions other than the active ingredient that are not toxic to the subject. Pharmacologically acceptable carriers include, but are not limited to, buffers, excipients, stabilizers, or preservatives.

A first aspect of the present disclosure provides an anti-PD-1 single-domain antibody. Single-domain antibodies usually refer to a class of antibody molecules that lack the light chains and only have the heavy chain variable regions. Antigen-binding properties of an antibody are usually determined by three complementarity determining regions (CDRs), which can be arranged in an orderly manner along with framework regions (FRs). The FRs do not directly participate in binding reactions. These CDRs can form loop structures and are brought close to each other in the spatial structure through β-sheets formed by the FRs, forming antigen-binding sites of the antibody. The CDRs of the anti-PD-1 single-domain antibody may include CDR1 having an amino acid sequence as set forth in one of SEQ ID Nos. 5 to 6, CDR2 having an amino acid sequence as set forth in one of SEQ ID Nos. 9 to 12, and CDR3 having an amino acid sequence as set forth in one of SEQ ID Nos. 17 to 21.

In a specific embodiment of the present disclosure, the CDRs of the anti-PD-1 single-domain antibody includes CDR1, which has an amino acid sequence as set forth in SEQ ID No. 5, CDR2, which has an amino acid sequence as set forth in SEQ ID No. 9, and CDR3, which has an amino acid sequence as set forth in SEQ ID No. 17.

In another specific embodiment of the present disclosure, the CDRs of the anti-PD-1 single-domain antibody includes CDR1, which has an amino acid sequence as set forth in SEQ ID No. 5, CDR2, which has an amino acid sequence as set forth in SEQ ID No. 10, and CDR3, which has an amino acid sequence as set forth in SEQ ID No. 18.

In another specific embodiment of the present disclosure, the CDRs of the anti-PD-1 single-domain antibody includes CDR1, which has an amino acid sequence as set forth in SEQ ID No. 6, CDR2, which has an amino acid sequence as set forth in SEQ ID No. 11, and CDR3, which has an amino acid sequence as set forth in SEQ ID No. 19.

In another specific embodiment of the present disclosure, the CDRs of the anti-PD-1 single-domain antibody includes CDR1, which has an amino acid sequence as set forth in SEQ ID No. 6, CDR2, which has an amino acid sequence as set forth in SEQ ID No. 10, and CDR3, which has an amino acid sequence as set forth in SEQ ID No. 18.

In another specific embodiment of the present disclosure, the CDRs of the anti-PD-1 single-domain antibody includes CDR1, which has an amino acid sequence as set forth in SEQ ID No. 6, CDR2, which has an amino acid sequence as set forth in SEQ ID No. 12, and CDR3, which has an amino acid sequence as set forth in SEQ ID No. 20.

In another specific embodiment of the present disclosure, the CDRs of the anti-PD-1 single-domain antibody includes CDR1, which has an amino acid sequence as set forth in SEQ ID No. 6, CDR2, which has an amino acid sequence as set forth in SEQ ID No. 9, and CDR3, which has an amino acid sequence as set forth in SEQ ID No. 21.

The anti-PD-1 single-domain antibody further includes framework regions (FRs), the FRs include FR1 having an amino acid sequence as set forth in one of SEQ ID No. 1 to 3, FR2 having an amino acid sequence as set forth in SEQ ID No. 7, FR3 having an amino acid sequence as set forth in one of SEQ ID No. 13 to 15 and SEQ ID No. 28 to 29, and FR4 having an amino acid sequence as set forth in one of SEQ ID No. 31 to 32.

In a specific embodiment of the present disclosure, the FRs includes FR1, which has an amino acid sequence as set forth in SEQ ID No. 1, FR2, which has an amino acid sequence as set forth in SEQ ID No. 7, FR3, which has an amino acid sequence as set forth in amino acid sequence SEQ ID No. 13, and FR4, which has an amino acid sequence as set forth in SEQ ID No. 31.

In another specific embodiment of the present disclosure, the FRs includes FR1, which has an amino acid sequence as set forth in SEQ ID No. 1, FR2, which has an amino acid sequence as set forth in SEQ ID No. 7, FR3, which has an amino acid sequence as set forth in amino acid sequence SEQ ID No. 14, and FR4, which has an amino acid sequence as set forth in SEQ ID No. 32.

In another specific embodiment of the present disclosure, the FRs includes FR1, which has an amino acid sequence as set forth in SEQ ID No. 1, FR2, which has an amino acid sequence as set forth in SEQ ID No. 7, FR3, which has an amino acid sequence as set forth in amino acid sequence SEQ ID No. 15, and FR4, which has an amino acid sequence as set forth in SEQ ID No. 31.

In another specific embodiment of the present disclosure, the FRs includes FR1, which has an amino acid sequence as set forth in SEQ ID No. 1, FR2, which has an amino acid sequence as set forth in SEQ ID No. 7, FR3, which has an amino acid sequence as set forth in amino acid sequence SEQ ID No. 28, and FR4, which has an amino acid sequence as set forth in SEQ ID No. 31.

In another specific embodiment of the present disclosure, the FRs includes FR1, which has an amino acid sequence as set forth in SEQ ID No. 2, FR2, which has an amino acid sequence as set forth in SEQ ID No. 7, FR3, which has an amino acid sequence as set forth in amino acid sequence SEQ ID No. 28, and FR4, which has an amino acid sequence as set forth in SEQ ID No. 31.

In another specific embodiment of the present disclosure, the FRs includes FR1, which has an amino acid sequence as set forth in SEQ ID No. 3, FR2, which has an amino acid sequence as set forth in SEQ ID No. 7, FR3, which has an amino acid sequence as set forth in amino acid sequence SEQ ID No. 29, and FR4, which has an amino acid sequence as set forth in SEQ ID No. 31.

The anti-PD-1 single-domain antibody provided by the present disclosure can be selected from: a) single-domain antibodies, each having an amino acid sequence including an amino acid sequence as set forth in one of SEQ ID Nos. 34 to 39; and b) single-domain antibodies, each having an amino acid sequence with a sequence identity of over 80% with an amino acid sequence as set forth in one of SEQ ID Nos. 34 to 39 and having functions of the single-domain antibodies as defined in a). Specifically, the single-domain antibodies in b) refer to: single-domain antibodies that are obtained by substitution, deletion or addition of one or more (specifically 1-50, 1-30, 1-20, 1-10, 1-5, or 1-3) amino acids on the basis of an amino acid sequence as set forth in one of SEQ ID No. 34-39, or obtained by adding one or more (specifically 1-50, 1-30, 1-20, 1-10, 1-5, or 1-3) amino acids at the N-terminus and/or C-terminus of this amino acid sequence, that have functions of a single-domain antibody having an amino acid sequence as set forth in one of SEQ ID No. 34-39. For example, the single-domain antibodies in b) can have a specific binding ability to PD-1 and can therefore specifically bind to cells expressing PD-1. For example, they can also block the interaction between PD-L1 and PD-1, thereby blocking the PD-L1/PD1 pathway (for example, blocking the binding of PD-1/PD-L1 on surfaces of effector cells and target cells), or increase the expression of IFN-γ and/or IL-2 in T lymphocytes, or inhibit tumor growth. The amino acid sequences of the single-domain antibodies in the b) can have 80%, 85%, 90%, 93%, 95%, 97%, or 99% or more identity with one of SEQ ID Nos. 42 to 49.

The anti-PD-1 single-domain antibody provided by the present disclosure can be derived from alpacas (Vicugna pacos), and its overall molecular weight can be about one-half of that of a single-chain antibody (scFv), thus effectively reducing the molecular weight of the overall structure, thereby enhancing its tissue penetration, enabling it to reach target tissues and organs more effectively, and improving the therapeutic effect. Also, it is more convenient to prepare the anti-PD-1 single-domain antibodies than structures with two scFv connected in tandem.

The anti-PD-1 single-domain antibody provided by the present disclosure is, for example, a humanized antibody. Humanized antibodies can be obtained from the aforementioned single-domain antibodies that are then humanized, thereby further improving their drug safety and effectively reducing the immunogenicity risks of the antibodies. Fully-humanized heavy-chain antibodies often suffer from poor solubility leading to protein aggregation and thus unsuitable for clinical practices. The decreased solubility of humanized single-domain antibodies may be due to the lack of a naturally paired light chain for the VHH domain of Ig-like monoclonal antibodies (Front Immunol. 2017 Nov. 22, 8:1603). In contrast, the humanized antibodies provided by the present disclosure, which were modified with the framework region, still maintain high solubility, making them possible to be used in clinical practice. The FRs of the humanized anti-PD-1 single-domain antibody include FR1 to FR4, whose amino acid sequences are as follows: FR1 has an amino acid sequence as set forth in SEQ ID No. 4, FR2 has an amino acid sequence as set forth in one of SEQ ID Nos. 7-8, FR3 has an amino acid sequence as set forth in amino acid sequence SEQ ID No. 16, and FR4 has an amino acid sequence as set forth in SEQ ID No. 33.

In a specific embodiment of the present disclosure, the FRs includes FR1, which has an amino acid sequence as set forth in SEQ ID No. 4, FR2, which has an amino acid sequence as set forth in SEQ ID No. 7, FR3, which has an amino acid sequence as set forth in amino acid sequence SEQ ID No. 16, and FR4, which has an amino acid sequence as set forth in SEQ ID No. 33.

In another specific embodiment of the present disclosure, the FRs includes FR1, which has an amino acid sequence as set forth in SEQ ID No. 4, FR2, which has an amino acid sequence as set forth in SEQ ID No. 8, FR3, which has an amino acid sequence as set forth in amino acid sequence SEQ ID No. 16, and FR4, which has an amino acid sequence as set forth in SEQ ID No. 33.

The anti-PD-1 single-domain antibody provided by the present disclosure can be a humanized anti-PD-1 single domain antibody and can be selected from: c) single-domain antibodies, each having an amino acid sequence including an amino acid sequence as set forth in one of SEQ ID Nos. 40 to 51; and d) single-domain antibodies, each having an amino acid sequence with a sequence identity of over 80% with an amino acid sequence as set forth in one of SEQ ID Nos. 40 to 51 and having functions of the single-domain antibodies as defined in c). Specifically, the single-domain antibodies in d) refer to: single-domain antibodies that are obtained by substitution, deletion or addition of one or more (specifically 1-50, 1-30, 1-20, 1-10, 1-5, or 1-3) amino acids on the basis of an amino acid sequence as set forth in one of SEQ ID No. 40-51, or obtained by adding one or more (specifically 1-50, 1-30, 1-20, 1-10, 1-5, or 1-3) amino acids at the N-terminus and/or C-terminus of this amino acid sequence, that have functions of a single-domain antibody having an amino acid sequence as set forth in one of SEQ ID No. 40-51. For example, the said functions can be either the specific binding ability to PD-1, enabling specific binding to PD-1-expressing cells, or the blockade of PD-1/PD-1 interaction, thus blocking the PD-L1/PD-1 pathway (for example, blocking the binding of PD-1/PD-L1 on the surface of effector cells and target cells). It can also involve enhancing IFN-γ and/or IL-2 expression in T lymphocytes, inhibiting tumor growth. The amino acid sequences of the single-domain antibodies in d) can have 80%, 85%, 90%, 93%, 95%, 97%, or 99% or more identity with one of SEQ ID Nos. 40 to 51.

A second aspect of the present disclosure provides a fusion protein including a first domain and a second domain, wherein the first domain includes the anti-PD-1 single-domain antibody, and the second domain includes a domain capable of prolonging the in vivo half-life of the fusion protein and/or a domain capable of binding to effector cells. The fusion protein described above may be a binding molecule and thus able to bind specifically to cells expressing PD-1.

In the fusion protein provided by the present disclosure, the domain capable of prolonging the in vivo half-life of the fusion protein may include one or more of serum albumin (e.g., human-derived HSA, etc.) or a fragment thereof, a domain that binds serum albumin (e.g., an anti-serum albumin antibody, including a single-domain antibody), polyethylene glycol, and a polyethylene glycol-liposome complex. The domain capable of binding to effector cells may include, for example, an immunoglobulin Fc region, and the immunoglobulin Fc region may preferably be a human immunoglobulin Fc region. The human immunoglobulin Fc region may further include mutations for eliminating, attenuating, or enhancing Fc-mediated effector functions, wherein the effector functions include one or more of CDC activity, ADCC activity, and ADCP activity. The immunoglobulin is one or more of IgG, IgA1, IgA2, IgD, IgE, and IgM, and the IgG is one or more of IgG1, IgG2, IgG3, and IgG4 isoforms. The immunoglobulin Fc region included in the single-domain antibody fusion protein may enable the fusion protein to form a dimer while extending the in vivo half-life of the fusion protein and increasing Fc-mediated correlated activity. The immunoglobulin Fc region can be the Fc region of human IgG1, more specifically it can be the wild-type IgG1 Fc sequence. The above sequences can be introduced with mutations to eliminate, weaken, or enhance Fc-mediated effector functions, for example, i) mutations to eliminate, weaken or enhance Fc-mediated CDC activity; or ii) mutations to eliminate, weaken or enhance Fc-mediated ADCC activity; or iii) mutations to eliminate, weaken or enhance Fc-mediated ADCP activity. Such mutations are described in: Leonard G Presta, Current Opinion in Immunology 2008, 20:460-470; Esohe E. Idusogie et al., J Immunol 2000, 164:4178-4184; RAPHAEL A. CLYNES et al., Nature Medicine, 2000, Volume 6, Number 4:443-446; Paul R. Hinton et al., J Immunol, 2006, 176:346-356. In a specific embodiment of the present disclosure, the immunoglobulin Fc region has an amino acid sequence set forth in one of SEQ ID No. 52 to 53, and SEQ ID No. 74 to 79.

The fusion protein provided by the present disclosure may also have a linker peptide between the first domain and the second domain. The linker peptide can be a flexible polypeptide consisting of glycine (G) and/or serine (S) and/or alanine (A) and/or threonine (T) of suitable length, capable of maintaining proper folding of the respective domains of the bispecific antibody molecule, as well as the flexibility between them, and the length of the linker peptide can be 3 to 30, 3 to 6, 6 to 9, 9 to 12, 12 to 16, 16 to 20, 20 to 25, 25 to 30, 8, or 15 amino acids. For example, the amino acid sequence of the linker peptide may include sequences such as (GS)n, (GGS)n, (GGSG)n, (GGGS)nA, (GGGGS)nA, (GGGGA)nA, (GGGGG) nA, wherein n is an integer from 0 to 10, for example, 0, 1, 2, 3, 4, 5, 6, 7, 8, 9, or 10.

In the fusion protein provided by the present disclosure, from the N-terminus to the C-terminus, it can sequentially include the first domain and the second domain. In a specific embodiment of the present disclosure, the amino acid sequence of the fusion protein includes an amino acid sequence as set forth in one of SEQ ID Nos. 58 to 69.

A third aspect of the present disclosure provides an isolated polynucleotide encoding the anti-PD-1 single-domain antibody as provided in the first aspect of the present disclosure, or the fusion protein as provided in the second aspect of the present disclosure. The above polynucleotides may be RNA, DNA, or cDNA, etc. Methods of providing the above isolated polynucleotides should be known to those skilled in the art; they can be obtained by, for example, automated DNA synthesis and/or recombinant DNA technologies, or they can be isolated from suitable natural sources.

A fourth aspect of the present disclosure provides a construct including the isolated polynucleotide provided in the third aspect of the present disclosure. The construction method of the above-mentioned construct should be known to those skilled in the art. For example, the above-mentioned construct can be constructed by methods such as in vitro recombinant DNA technologies, DNA synthesis technologies, and in vivo recombination technologies. More specifically, the above-mentioned constructs can be constructed by inserting the above-mentioned isolated polynucleotides into a suitable vector (for example, a multiple cloning site of the vector). The person skilled in the art may select a suitable vector for the construction of the above constructs. For example, the vector can be a bacterial plasmid, phage, yeast plasmid, plant cell virus, mammalian cell virus such as adenovirus, retrovirus, or any other suitable vectors. For instance, the vector can be an expression vector or a cloning vector. The vector can also include one or more regulatory sequences operatively linked to the above polynucleotide sequences. A regulatory sequence can usually include a suitable promoter sequence, transcription terminator sequence, enhancer sequence, etc., as well as replication origin, convenient restriction enzyme sites and one or more selectable markers. The promoter sequence is typically operatively linked to the coding sequence of the amino acid sequence to be expressed. The promoters can be any nucleotide sequence that exhibits transcriptional activity in the selected host cell, including mutated, truncated, and chimeric promoters and can be obtained from genes encoding extracellular or intracellular peptides that are homologous or heterologous to the host cell. For example, these promoters can be: E. coli's lac or trp promoters; λ phage PL promoter; eukaryotic promoters including CMV immediate early promoter, HSV thymidine kinase promoter, early and late SV40 promoter, Pichia pastoris methanol oxidase promoter; and other known promoters that control gene expression in prokaryotic or eukaryotic cells or their viruses. The regulatory sequence can also include suitable transcription terminator sequences recognized by the host cell to terminate transcription. The terminator sequence is connected to the 3′ end of the nucleotide sequence encoding the peptide and any terminator functional in the selected host cell can be used in the present disclosure. Marker genes can be used for selecting phenotypic traits of transformed host cells, and may include, for example, dihydrofolate reductase for eukaryotic cell culture, neomycin resistance and green fluorescent protein (GFP), or tetracycline or ampicillin resistance for E. coli. When the polynucleotides described above are to be expressed, the expression vector may also include an enhancer sequence that, if inserted into the vector, will result in enhanced transcription. Enhancers are cis-acting factors of DNA, typically of about 10 to 300 base pairs, that act on promoters to enhance gene transcription.

A fifth aspect of the present disclosure provides an expression system for an antibody; the expression system includes the construct provided in the fourth aspect of the present disclosure, or a genome incorporating an exogenous polynucleotide provided in the third aspect of the present disclosure, thereby allowing expression of the anti-PD-1 single-domain antibody, or the fusion protein. Any cell line suitable for expression by an expression vector can serve as a host cell. For example, the host cell can be a prokaryotic cell, such as a bacterial cell; or a lower eukaryotic cell, such as a yeast cell; a filamentous fungal cell; or a higher eukaryotic cell, such as a mammalian cell. More specifically, it can be, for example, Escherichia co/i, Streptomyces spp; bacterial cells of Salmonella typhimurium; fungal cells such as yeast, filamentous fungi, plant cells; insect cells such as Drosophila S2 or Sf9; animal cells of CHO, COS, 293 cells, or Bowes melanoma cells, etc. Methods of constructing the expression system should be known to those skilled in the art; for example, they may include one or more of microinjection, gene gun, electroporation, virus-mediated transformation, electron bombardment, calcium phosphate precipitation.

A sixth aspect of the present disclosure provides a method for preparing the anti-PD-1 single-domain antibody provided in the first aspect of the present disclosure, or the fusion protein provided in the second aspect of the present disclosure, including: culturing the expression system for an antibody provided in the fifth aspect of the present disclosure under conditions suitable for expressing the antibody or the fusion protein, thereby expressing the antibody or the fusion protein; and purifying and isolating the antibody or the fusion protein.

A seventh aspect of the present disclosure provides use of the single-domain antibody provided in the first aspect of the present disclosure, or use of the fusion protein provided in the second aspect of the present disclosure, in the preparation of a drug and/or a medicament for the diagnosis and/or treatment of a disease associated with cells expressing PD-1. The single-domain antibody provided by the present disclosure can bind specifically to PD-1, thereby binding specifically to cells expressing PD-1, can block PD-L1/PD-1 interaction, and can also increase IFN-γ and/or IL-2 expression in T lymphocytes; therefore, they can be used in the preparation of relevant drugs and/or agents.

Specifically, diseases associated with cells expressing or not expressing PD-1 in the uses provided by the present disclosure may include one or more of cancers, tumors, etc., and more specifically, they may be, for example, lung cancer, melanoma, gastric cancer, ovarian cancer, colon cancer, liver cancer, kidney cancer, bladder cancer, breast cancer, classical Hodgkin's lymphoma, hematological malignancies, head and neck cancer, and/or nasopharyngeal cancer, which may be early, intermediate, or advanced; they may also be metastatic cancers.

A ninth aspect of the present disclosure provides a pharmaceutical composition including the anti-PD-1 single-domain antibody as provided in the first aspect of the present disclosure, the fusion protein as provided in the second aspect of the present disclosure, or a culture of the expression system as provided in the fifth aspect of the present disclosure. The amount of the anti-PD-1 single-domain antibody, fusion protein, or culture in the above-described pharmaceutical composition is typically a therapeutically effective amount. The above-described pharmaceutical composition may also include pharmaceutically acceptable carriers. These pharmaceutically acceptable carriers may include a variety of excipients and diluents that are not themselves essential active ingredients and are not unduly toxic upon administration. Suitable carriers should be well known to those skilled in the art, for example, a full discussion of pharmaceutically acceptable carriers can be found in Remington's Pharmaceutical Sciences (Mack Pub. Co., N.J., 1991). In a preferred specific embodiment of the present disclosure, the pharmaceutical composition can be administered by injection, so that the pharmaceutical composition is preferably a powder for injection (such as freeze-dried powder for injection) and/or a liquid agent.

In the above-mentioned drugs, or pharmaceutical composition, etc., the relevant substances (e.g., single-domain antibody, fusion protein, culture, etc.) may be a single active ingredient respectively, or may be combinations formed with other active components.

A tenth aspect of the present disclosure provides a therapeutic method including administering to an individual a therapeutically effective amount of the anti-PD-1 single-domain antibody provided in the first aspect of the present disclosure, the fusion protein provided in the second aspect of the present disclosure, the culture of the expression system of an antibody provided in the fifth aspect of the present disclosure, or the pharmaceutical composition provided in the ninth aspect of the present disclosure. The therapeutic method provided by the present disclosure can be used to treat tumors, or other indications. The selection of a preferred therapeutically effective amount can generally be determined by one of ordinary skill in the art based on various factors (e.g., through clinical trials), such as the fact that tumor growth, proliferation, recurrence and/or metastasis can be inhibited when the substance is used in the individual to whom it is administered, and more specifically, that at least 10%, 20%, 30%, 40%, 50%, 60%, 70%, 80%, 90%, 95%, or 99% of the tumor growth, proliferation, recurrence and/or metastasis is inhibited when the substance is used.

The anti-PD-1 single-domain antibody provided by the present disclosure has a specific binding ability for PD-1 and can be used to further construct fusion proteins, etc, which can also increase IFN-γ and/or IL-2 expression in T lymphocytes and can effectively inhibit tumor growth, and therefore has good prospects for industrialization.

The present disclosure is further described below by means of embodiments, but the scope of the present disclosure is not thereby limited.

Unless otherwise stated, the experimental methods, detection methods, and preparation methods disclosed in the present disclosure are conventional techniques in molecular biology, biochemistry, chromatin structure and analysis, analytical chemistry, cell culture, recombinant DNA technology, and related fields in the art. These techniques are well described in the existing literature, see Sambrook et al, MOLECULAR CLONING: A LABORATORY MANUAL, Second edition, Cold Spring Harbor Laboratory Press, 1989 and Third edition, 2001; Ausubel et al, CURRENT PROTOCOLS IN MOLECULAR BIOLOGY, John Wiley & Sons, New York, 1987 and periodic updates; the series METHODS IN ENZYMOLOGY, Academic Press, San Diego; Wolffe, CHROMATIN STRUCTURE AND FUNCTION, Third edition, Academic Press, San Diego, 1998; METHODS IN ENZYMOLOGY, Vol. 304, Chromatin (P. M. Wassarman and A. P. Wolffe, eds.), Academic Press, San Diego, 1999; and METHODS IN MOLECULAR BIOLOGY, Vol. 119, Chromatin Protocols (P. B. Becker, ed.) Humana Press, Totowa, 1999, etc.

### Embodiment 1

Construction of Anti-PD1 Single-Domain Antibody Library

1 mg of a fusion protein hPD1-HSA (SEQ ID NO:54) composed of a PD1 extracellular domain sequence and a human serum albumin sequence was mixed and emulsified with 1 ml of Freund's complete adjuvant (Sigma), and healthy alpacas (Vicugna pacos) were immunized with the mixture. The alpacas were immunized again after twenty-one days, for a total of three immunizations, to stimulate B cells to express antigen-specific single-domain antibodies. One week after the 3 immunizations, 30 ml of alpaca blood was collected using a vacuum blood collection tube. Lymphocytes were separated using lymphocyte separation solution (Tianjin Haoyang Huake Biotechnology Co., Ltd.) and total RNA was extracted using the Trizol method. Using a reverse transcription kit (Invitrogen), 3 μg of total RNA was reverse transcribed into cDNA according to the instructions, and VHH was amplified using nested PCR. The target VHH nucleic acid fragment was recovered and digested with the restriction endonuclease SfiI (NEB), and inserted into the phage display vector pcomb3xss (Addgene plasmid #63890; RRID: Addgene_63890) that was also digested with the same enzyme, and connected using T4 ligase (Takara). The ligation product was transformed into electrocompetent cells ER2738 to construct an anti-PD1 single-domain antibody library. The library size was determined to be 1.5×108 by gradient dilution plating. At the same time, 25 clones were randomly selected for colony PCR detection, and the results showed that the insertion rate of the constructed library was 100%.

### Embodiment 2

Screening and Identification of Anti-PD-1 Single-Domain Antibody

2.1 Screening of Anti-PD-1 Single-Domain Antibody

The constructed anti-PD1 single-domain antibody library was packaged using helper phage M13KO7 (NEB) and the recombinant phage titer of displayed library was determined to be 8.5×10¹² PFU/ml. hPD1-HSA was diluted to 2 μg/ml with coating buffer of 100 mM NaHCO₃ (pH 8.2), and 100 μL/well of the diluted product was added to an enzyme-linked immunosorbent assay (ELISA) plate and incubated overnight at 4° C. The next day, the plate was washed 5 times with PBST (PBS containing 0.1% Tween-20), blocked with 200 μL of 3% BSA at 37° C. for 1 hour, and then incubated with 100 μL of recombinant phages (approximately 1.5×10¹¹ PFU/well, from the library constructed in Embodiment 1) at room temperature for 1 hour. After that, the ELISA plates were washed 5 times with PBST to remove non-specific binding phage. The washed ELISA plate wells were incubated with 1 ml of 0.1 M gly-HCl+1 mg/ml BSA (pH 2.2) buffer for 10 min to elute and neutralized with 1 M pH 8.0 Tris-HCl. The phage titer was determined to be 4.45×10⁶ PFU/ml. The phage eluate was amplified and the titer thereof was determined to be 1.6×10¹³ PFU/ml. Using the fusion protein hPD1-Fc (SEQ ID NO:56) composed of the PD-1 extracellular domain sequence and human IgG1 FC as a coating protein and blocked with 3% ovalbumin (OVA), a second round of screening was performed using the same screening process, and the phage titer of the second round of elution was determined to be 1.96×108 PFU/ml.

2.2 Dual Screening by Enzyme-Linked Immunoassay (ELISA) and TEPITOPE

400 single clones were picked from the phage titer assay plate of the second round of elution, then cultured in a 96-well plate, and then infected and packaged with helper phage M13KO7 to obtain the accumulation of recombinant phages in the supernatant.

hPD1-Fc was coated at 200 ng/well and blocked with skim milk powder at room temperature for 1 hour. The supernatant of monoclonal recombinant phages was diluted twofold with PBS and 100 ul/well of the diluted supernatant was added and incubated together at 37° C. for 1 hour. After washing 3 times with PBST, 100 μl of Anti-M13 Antibody (HRP) (Sino Biological Inc.) diluted 1:10000 was added to each well and incubated at 37° C. for 1 hour. After washing 3 times with PBST, TMB color development working solution (Beijing ComWin Biotech Co., Ltd.) was added and incubated at room temperature for 5 minutes to develop color. The reaction was terminated by adding 1M sulfuric acid and OD450 nm reading was recorded. Most of the OD450 nm values were greater than 3, indicating positive binding.

After mixing the monoclonal recombinant phage supernatant with 4 μg/mL Bio-hPDL1-HSA (biotin-labeled, self-made) in a 96-well plate in equal proportions, 100 μl/well of the mixture was incubated in a 96-well plate pre-coated with 100 ng hPD1-Fc at 37° C. for 1 hour; a well containing Bio-hPDL1-HSA (diluted to 2 μg/mL with PBS) without recombinant phage supernatant was used as a control well, and incubated at 37° C. for 1 hour. After washing 3 times with PBST, 100 μl of Streptavidin-HRP diluted 1:50000 (Jackson ImmunoResearch Inc) was added to each well and incubated at 37° C. for 1 hour. After washing 3 times with PBS, TMB color development working solution (Beijing ComWin Biotech Co., Ltd.) was added and incubated at room temperature for 5 minutes to develop color, then 1M sulfuric acid was added to terminate the reaction and OD450 nm reading was recorded. The control well showed obvious coloration (whose OD450 nm value was 1.16), and competitive positive phage clones showed weak or basically no coloration (OD450 nm values less than 0.2). Competitive positive clones were selected for sequencing and duplicate sequences were removed.

Further immunogenicity risk analysis was performed on the CDR3 sequences of the obtained high-affinity positive clones. Scores of alleles DRB1*03:01, DRB1*07:01, DRB1*15:01, DRB3*01:01, DRB3*02:02, DRB4*01:01 and DRB5*01:01 closely related to immunogenicity risk in human were calculated using the prediction tool TEPITOPE (Sturniolo T et al., Nat. Biotechnol. 17:555-561) based on the QAM method. Sequences with a total CDR3 TEPITOPE score higher than 4 were excluded and highly humanized anti-PD-1 single-domain antibody sequences were obtained after further screening. Most of the CDR3 TEPITOPE total scores of the anti-PD-1 single-domain antibodies finally obtained by the present disclosure were <−2.0, which were significantly lower than those of similar anti-PD-1 single-domain antibodies, indicating that the anti-PD-1 single-domain antibodies of the present disclosure have lower potential immunogenicity risk.

After several rounds of screening, the inventors finally found six anti-PD1 single-domain antibody clones showing strongly competitive positivity and low TEPITOPE scores. The full-length sequences are shown in Table 1a, where the underlined portions are CDR regions labeled by the IMGT method.

TABLE 1a

| Clone | | |
|---|---|---|
| code | Full-length sequence | SEQ ID No. |
| 1A4 | QVQLVESGGGLVQPGGSLNLSCVASGNVFIIDVMAWYRQAPGKQRELVAQIING | 34 |
| | DAKYYVDSVKGRFTISRDNTKTTLYLQMNSLKPDDTAIYYCSAEKFNPY | |
| | RDSTNFWGQGTQVTVSS | |
| 1A9 | QVQLVESGGGLVQPGGSLNLSCVASGNVFIIDVMAWYRQAPGKQRELVAQIING | 35 |
| | GATYYADSVKGRFTISRDDTKTTLNLQMNSLKPDDTAVYYCSAEKFNPYRDST | |
| | HFWGQGTQVTVSA | |
| 1H1 | QVQLVESGGGLVQPGGSLNLSCVASGSVFIIDVMAWYRQAPGKQRELVAQIING | 36 |
| | GASYYADSVKGRFTISRDNTKTTLFLQMNSLKPADTAVYYCNAEKFNPYRDSTN | |
| | FWGQGTQVTVSS | |
| 1D3 | QVQLVESGGGLVQPGGSLNLSCVASGSVFIIDVMAWYRQAPGKQRELVAQIING | 37 |
| | GATYYADSVKGRFTISRDDTKTTLYLQMNSLKPDDTAVYYCSAEKFNPYRDSTH | |
| | FWGQGTQVTVSS | |
| 2C4 | QVQLVESGGGLVRPGGSLRLSCAASGSVFIIDVMAWYRQAPGKQRELVAQIISG | 38 |
| | DHAYYADSVKGRFTISRDDTKTTLYLQMNSLKPDDTAVYYCSAEKFNPYRDSTY | |
| | FWGQGTQVTVSS | |
| 2D9 | QVQLVESGGGLVQPGGSLRLSCAASGSVFIIDVMAWYRQAPGKQRELVAQIING | 39 |
| | DAKYYADSVKGRFTISRDNTKTTLYLQMNSLKPDDTAVYYCSAEQFNPYRDST | |
| | YFWGQGTQVTVSS | |

The framework regions (FRs) and complementary determining regions (CDRs) of each antibody are shown in Table 1 b.

TABLE 1b

| | FR1 | CDR1 | FR2 | CDR2 | FR3 | CDR3 | FR4 |
|---|---|---|---|---|---|---|---|
| Clone | (SEQ | (SEQ | (SEQ | (SEQ | (SEQ | (SEQ | (SEQ |
| code | ID No.) | ID No.) | ID No.) | ID No.) | ID No.) | ID No.) | ID No.) |
| 1A4 | 1 | 5 | 7 | 9 | 13 | 17 | 31 |
| 1A9 | 1 | 5 | 7 | 10 | 14 | 18 | 32 |
| 1H1 | 1 | 6 | 7 | 11 | 15 | 19 | 31 |
| 1D3 | 1 | 6 | 7 | 10 | 28 | 18 | 31 |
| 2C4 | 2 | 6 | 7 | 12 | 28 | 20 | 31 |
| 2D9 | 3 | 6 | 7 | 9 | 29 | 21 | 31 |

CDR3 TEPITOPE scores are shown in Table 1c.

TABLE 1c

| | CDR3 | | | | | | | | |
|---|---|---|---|---|---|---|---|---|---|
| | SEQ | TEPITOPE score (Threshold: 3%) | | | | | | | Total |
| Clone code | ID No. | DRB1*03:01 | DRB1*07:01 | DRB1*15:01 | DRB3*01:01 | DRB3*02:02 | DRB4*01:01 | DRB5*01:01 | score |
| 1A4 | 17 | −4.0304 | −2.4215 | 1.2525 | −2.9365 | −3.4986 | −4.2453 | −1.7192 | −17.599 |
| 1A9 | 18 | −3.6839 | −2.1215 | 1.3755 | −1.9048 | −2.1077 | −2.8816 | −0.7259 | −12.0499 |
| 1H1 | 19 | −4.0304 | −2.4215 | 1.2525 | −2.9365 | −3.4986 | −4.2453 | −1.7192 | −17.599 |
| 2C4 | 20 | −3.2317 | −0.8215 | 1.5705 | −2.544 | −3.1271 | −3.8779 | −0.4251 | −12.4568 |
| 2D9 | 21 | −3.2317 | −0.8215 | 1.5705 | −2.544 | −3.1271 | −3.8779 | −0.4251 | 12.4568 |
| Control 1 | 70 | 4.3006 | 5.4723 | 3.9636 | 3.0847 | 1.0677 | 3.0646 | 2.5787 | 23.5322 |
| Control 2 | 71 | −1.6379 | 2.3656 | −1.4384 | −1.6004 | −1.9265 | −2.6357 | −1.1133 | −7.9866 |
| Control 3 | 72 | 1.5267 | 1.3741 | 2.1261 | −1.1655 | −2.1242 | −0.157 | 0.8815 | 2.4617 |

Note:
Control 1 is derived from the anti-PD-1 single-domain antibody shown in US2019/0322747A1 (SEQ ID No. 14). Control 2 is derived from the anti-PD-1 single-domain antibody shown in CN110256562A (SEQ ID No. 9). Control 3 is derived from the anti-PD-1 single-domain antibody shown in CN110003336A (SEQ ID No. 6).

### Embodiment 3

Preliminary Evaluation of Anti-PD-1 Single-Domain Antibodies

3.1 Expression and Purification of Single-Domain Antibodies in E. coli Host

Using the plasmid sequences of the specific positive clones obtained by screening as a template, PCR amplifications were performed using the high-fidelity enzyme GVP8 (General Biosystems (Anhui) Corporation Limited), and a histidine tag coding sequence was introduced at the 3′ end of the sequence. The PCR product was electrophoresed and gel-recovered to obtain a band of about 600 bp. The recovered PCR product was recombined with the pET32a+ vector (Novagen) digested with the restriction enzymes NdeI (NEB) and EcoRI (NEB) using a recombination kit (Novoprotein Scientific Inc) to construct an E. coli expression plasmid, which was then transformed into E. coli Top10F′ competent cells, plated on ampicillin-resistant plates, and incubated overnight at 37° C. Clones were picked from the ampicillin-resistant plates, whose plasmids were extracted and sequenced to confirm correct insertion of the sequence into the pET32a+ vector. E. coli expression plasmids confirmed by sequencing were transformed into the E. coli expression host Rosetta (DE3) to construct an E. coli expression strain. Recombinant clones were picked from the ampicillin-resistant plates, cultured, and induced to express overnight at 30° C. with 1 mM IPTG. The induced bacterial culture was sonicated and centrifuged at 12000 g for 10 minutes at 4° C. to obtain the supernatant. The supernatant was purified using a Ni column (Bestchrom (Shanghai) Biosciences Ltd) and the final protein purity reached over 90%.

3.2 Confirmation of Specific Binding of Purified Anti-PD-1 Single-Domain Antibody Recombinant Protein to Human and Mouse PD-1

Human hPD1-HSA, mouse mPD1-HSA (SEQ ID NO: 57) or HSA (purchased from Sigma) were coated on plates at 200 ng/well overnight at 4° C. and blocked with 5% skim milk at room temperature for 1 hour. Purified histidine-tagged anti-PD-1 single-domain antibody was diluted to 1 μg/mL and incubated at 37° C. for 1 hour with 100 μL per well. After washing with PBST 3 times, a mouse anti-his tag antibody (R&D Systems, Inc) diluted 1:5000 was added at 100 μl/well and incubated at room temperature for 1 hour. After washing with PBST, HRP-Goat anti-mouse IgG antibody (Thermo Scientific) diluted 1:10000 was added at 100 μl/well and incubated at room temperature for 1 hour. After washing with PBST, TMB color developing solution (Beijing ComWin Biotech Co., Ltd.) was added for color development and OD value was measured at 450 nm.

The results are shown in Table 2. The results show that the six clones obtained above specifically bind to human PD-1 and exhibit good binding activity with human PD-1. When coated with mouse mPD1-HSA fusion protein, the OD values of the anti-PD-1 single-domain antibodies were not significantly different from those of the negative control group without the addition of anti-PD-1 single-domain antibodies, indicating that none of the six clones bind to mouse PD-1 (results not shown).

TABLE 2

| | OD450 nm | OD450 nm |
|---|---|---|
| Clone code | (hPD1-HSA coated on plate) | (HSA coated on plate) |
| 1A4 | 2.132 | 0.021 |
| 1A9 | 2.119 | 0.035 |
| 1H1 | 2.175 | 0.031 |
| 1D3 | 2.098 | 0.029 |
| 2C4 | 2.141 | 0.022 |
| 2D9 | 2.109 | 0.019 |

3.3 Confirmation of Specific Binding of Purified Anti-PD-1 Single-Domain Antibody Recombinant Protein to Monkey PD-1

Monkey RhPD1-HSA fusion protein (SEQ ID No. 30) was coated on plates at 200 ng/well overnight at 4° C. and blocked with 5% skim milk at room temperature for 1 hour. Purified histidine-tagged anti-PD1 single-domain antibody was diluted to 1 μg/mL and incubated at 37° C. for 1 hour with 100 μL per well. After washing with PBST 3 times, a mouse anti-his tag antibody (R&D Systems, Inc) diluted 1:5000 was added at 100 μl/well and incubated at room temperature for 1 hour. After washing with PBST, HRP-Goat anti-mouse IgG antibody (Thermo Scientific) diluted 1:10000 was added at 100 μl/well and incubated at room temperature for 1 hour. After washing with PBST, TMB color developing solution was added for color development and OD value was measured at 450 nm.

The results showed that the OD values of the anti-PD1 single-domain antibodies were significantly different from those of the negative control (Blank) without the addition of anti-PD1 single-domain antibody, indicating that all six clones screened bind to monkey PD-1.

TABLE 3

| | OD450 nm |
|---|---|
| Clone code | (RhPD1-HSA coated on plate) |
| 1A4 | 2.003 |
| 1A9 | 2.019 |
| 1H1 | 2.025 |
| 1D3 | 2.101 |
| 2C4 | 2.034 |
| 2D9 | 2.105 |
| Blank | 0.132 |

### Embodiment 4

Humanization of Anti-PD-1 Single-Domain Antibodies

The VHH humanization framework transplantation (Vincke C, Loris R, Saerens D, Martinez-Rodriguez S, Muyldermans S, Conrath K. J Biol Chem. 2009; 284(5):3273-3284) was used in humanization. Based on sequence homology, a universal humanized VHH framework h-NbBcII10FGLA (PDB ID: 3EAK) was designed. The corresponding CDR regions were replaced with the CDR regions of the anti-PD-1 single-domain antibody, and individual amino acids in the FR2 region were further humanized according to the sequence of the humanized antibody DP-47 (SEQ ID NO:73). Multiple humanized variants were obtained for each anti-PD-1 single-domain antibody, and a humanized scheme with high solubility and low aggregation formation was selected while introducing as many human amino acids as possible. The humanized single-domain antibodies were expressed and purified according to Embodiment 3.1.

The preferred amino acid sequences of humanized VHH are SEQ ID NO:40 to 51, as shown in Table 4a, where CDR regions are underlined.

TABLE 4a

| | | | Nucleo- |
|---|---|---|---|
| | | | tide |
| Human- | | | Se- |
| ized | | SEQ | quences |
| clone | | ID | SEQ ID |
| codes | Full-length sequences | No. | No. |
| hu1A4V1 | QVQLVESGGGLVQPGGSLRLSCAAS | 40 | 80 |
| | GNVFIIDVMAWYRQAPGKQRELVAQ | | |
| | IINGDAKYYADSVKGRFTISRDNSK | | |
| | NTLYLQMNSLRAEDTAVYYC | | |
| | SAEKFNPYRDSTNFWGQGTLVTVSS | | |
| hu1A9V1 | QVQLVESGGGLVQPGGSLRLSCAAS | 41 | 81 |
| | GNVFIIDVMAWYRQAPGKQRELVAQ | | |
| | IINGGATYYADSVKGRFTISRDNSK | | |
| | NTLYLQMNSLRAEDTAVYYC | | |
| | SAEKFNPYRDSTHFWGQGTLVTVSS | | |
| hu1H1V1 | QVQLVESGGGLVQPGGSLRLSCAAS | 42 | 82 |
| | GSVFIIDVMAWYRQAPGKQRELVAQ | | |
| | IINGGASYYADSVKGRFTISRDNSK | | |
| | NTLYLQMNSLRAEDTAVYYC | | |
| | NAEKFNPYRDSTNFWGQGTLVTVSS | | |
| hu1D3V1 | QVQLVESGGGLVQPGGSLRLSCAAS | 43 | 83 |
| | GSVFIIDVMAWYRQAPGKQRELVAQ | | |
| | IINGGATYYADSVKGRFTISRDNSK | | |
| | NTLYLQMNSLRAEDTAVYYC | | |
| | SAEKFNPYRDSTHFWGQGTLVTVSS | | |
| hu2C4V1 | QVQLVESGGGLVQPGGSLRLSCAAS | 44 | 84 |
| | GSVFIIDVMAWYRQAPGKQRELVAQ | | |
| | IISGDHAYYADSVKGRFTISRDNSK | | |
| | NTLYLQMNSLRAEDTAVYYC | | |
| | SAEKFNPYRDSTYFWGQGTLVTVSS | | |
| hu2D9V1 | QVQLVESGGGLVQPGGSLRLSCAAS | 45 | 85 |
| | GSVFIIDVMAWYRQAPGKQRELVAQ | | |
| | IINGDAKYYADSVKGRFTISRDNSK | | |
| | NTLYLQMNSLRAEDTAVYYC | | |
| | SAEQFNPYRDSTYFWGQGTLVTVSS | | |
| hu1A4V3 | QVQLVESGGGLVQPGGSLRLSCAAS | 46 | 86 |
| | GNVFIIDVMAWYRQAPGKGLELVAQ | | |
| | IINGDAKYYADSVKGRFTISRDNSK | | |
| | NTLYLQMNSLRAEDTAVYYC | | |
| | SAEKFNPYRDSTNFWGQGTLVTVSS | | |
| hu1A9V3 | QVQLVESGGGLVQPGGSLRLSCAAS | 47 | 87 |
| | GNVFIIDVMAWYRQAPGKGLELVAQ | | |
| | IINGGATYYADSVKGRFTISRDNSK | | |
| | NTLYLQMNSLRAEDTAVYYC | | |
| | SAEKFNPYRDSTHFWGQGTLVTVSS | | |
| hu1H1V3 | QVQLVESGGGLVQPGGSLRLSCAAS | 48 | 88 |
| | GSVFIIDVMAWYRQAPGKGLELVAQ | | |
| | IINGGASYYADSVKGRFTISRDNSK | | |
| | NTLYLQMNSLRAEDTAVYYC | | |
| | NAEKFNPYRDSTNFWGQGTLVTVSS | | |
| hu1D3V3 | QVQLVESGGGLVQPGGSLRLSCAAS | 49 | 89 |
| | GSVFIIDVMAWYRQAPGKGLELVAQ | | |
| | IINGGATYYADSVKGRFTISRDNSK | | |
| | NTLYLQMNSLRAEDTAVYYC | | |
| | SAEKFNPYRDSTHFWGQGTLVTVSS | | |
| hu2C4V3 | QVQLVESGGGLVQPGGSLRLSCAAS | 50 | 90 |
| | GSVFIIDVMAWYRQAPGKGLELVAQ | | |
| | IISGDHAYYADSVKGRFTISRDNSK | | |
| | NTLYLQMNSLRAEDTAVYYC | | |
| | SAEKFNPYRDSTYFWGQGTLVTVSS | | |
| hu2D9V3 | QVQLVESGGGLVQPGGSLRLSCAAS | 51 | 91 |
| | GSVFIIDVMAWYRQAPGKGLELVAQ | | |
| | IINGDAKYYADSVKGRFTISRDNSK | | |
| | NTLYLQMNSLRAEDTAVYYC | | |
| | SAEQFNPYRDSTYFWGQGTLVTVSS | | |

The framework regions (FRs) and complementary determining regions (CDRs) of each humanized variant are shown in Table 4b.

TABLE 4b

| | FR1 | CDR1 | FR2 | CDR2 | FR3 | CDR3 | FR4 |
|---|---|---|---|---|---|---|---|
| | (SEQ | (SEQ | (SEQ | (SEQ | (SEQ | (SEQ | (SEQ |
| Humanized | ID | ID | ID | ID | ID | ID | ID |
| clone codes | No.) | No.) | No.) | No.) | No.) | No.) | No.) |
| hu_{1A4V1} | 4 | 5 | 7 | 9 | 16 | 17 | 33 |
| hu_{1A9V1} | 4 | 5 | 7 | 10 | 16 | 18 | 33 |
| hu_{1H1V1} | 4 | 6 | 7 | 11 | 16 | 19 | 33 |
| hu_{1D3V1} | 4 | 6 | 7 | 10 | 16 | 18 | 33 |
| hu_{2C4V1} | 4 | 6 | 7 | 12 | 16 | 20 | 33 |
| hu_{2D9V1} | 4 | 6 | 7 | 9 | 16 | 21 | 33 |
| hu_{1A4V3} | 4 | 5 | 8 | 9 | 16 | 17 | 33 |
| hu_{1A9V3} | 4 | 5 | 8 | 10 | 16 | 18 | 33 |
| hu_{1H1V3} | 4 | 6 | 8 | 11 | 16 | 19 | 33 |
| hu_{1D3V3} | 4 | 6 | 8 | 10 | 16 | 18 | 33 |
| hu_{2C4V3} | 4 | 6 | 8 | 12 | 16 | 20 | 33 |
| hu_{2D9V3} | 4 | 6 | 8 | 9 | 16 | 21 | 33 |

The degree of humanization of each humanized single-domain antibody (by comparison of sequence identity with the closest human gene and allele) was analyzed by IMGT/3D structure-DB (website http://www.imgt.org), available in the literature: Use of IMGT® Databases and Tools for Antibody Engineering and Humanization, Marie-PauleLefranc et al, Methods Mol Biol, 907:3-37, 2012. The results are shown in Table 4c.

TABLE 4c

| | | Sequence |
|---|---|---|
| Codes | Closest human genes and alleles | identities (%) |
| hu1A4V1 | IGHV3-30*02, IGHJ1*01 | 80.4, 100 |
| hu1A9V1 | IGHV3-64*04, IGHJ1*01 | 81.4, 100 |
| hu1H1V1 | IGHV3-64*04, IGHJ1*01 | 81.2, 100 |
| hu1D3V1 | IGHV3-64*04, IGHJ1*01 | 81.4, 100 |
| hu2C4V1 | GHV3-23*04, IGHJ5*01 | 79.4, 100 |
| hu2D9V1 | IGHV3-30*02, IGHJ5*01 | 80.4, 100 |
| hu1A4V3 | IGHV3-30*02, IGHJ1*01 | 82.5, 100 |
| hu1A9V3 | IGHV3-64*04, IGHJ1*01 | 83.5, 100 |
| hu1H1V3 | IGHV3-64*04, IGHJ1*01 | 83.3, 100 |
| hu1D3V3 | IGHV3-64*04, IGHJ1*01 | 83.5, 100 |
| hu2C4V3 | GHV3-23*04, IGHJ5*01 | 81.4, 100 |
| hu2D9V3 | IGHV3-30*02, IGHJ5*01 | 82.5, 100 |
| Control 1 | IGHV3-23*01, IGHJ4*01 | 79.4, 92.9 |
| Control 2 | IGHV3-23*01, IGHJ4*01 | 82.3, 78.6 |
| Control 3 | IGHV3-23*01, IGHJ5*02 | 78.4, 90.9 |
| Bevacizumab | IGHV3-30*02, IGHJ4*01 | 76.8, 93.3 |
| HCDR3 | | |
| Adalimumab | IGHV3-9*01, IGHJ4*01 | 93.9, 92.9 |
| HCDR3 | | |

Note:
1) Control 1 is derived from the anti-PD-1 single-domain antibody shown in US2019/0322747A1 (SEQ ID No. 14). Control 2 is derived from the anti-PD-1 single-domain antibody shown in CN110256562A (SEQ ID No. 9). Control 3 is derived from the anti-PD-1 single-domain antibody shown in CN110003336A (SEQ ID No. 6).

(2) Bevacizumab HCDR3 represents the heavy chain CDR3 sequence of the marketed monoclonal antibody Bevacizumab, and Adalimumab HCDR3 is the heavy chain CDR3 sequence of the marketed monoclonal antibody Adalimumab. (3) When assigning codes to different anti-PD-1 single-domain antibodies, V1 and V3 represent two different humanized sequences.

### Embodiment 5

Preparation of Anti-PD1-Fc Fusion Protein from Mammalian Cells

5.1 Expression and Purification of Anti-PD-1 Single-Domain Antibody and Fc Fusion Proteins (Anti-PD1-Fc)

Specific positive sequences obtained by screening (SEQ ID NO:34-39) and humanized sequences (SEQ ID NO:40-51) were converted into corresponding nucleotide sequences according to the codon preference of CHO cells. Corresponding full-length DNA molecules were obtained by gene synthesis (GenScript Biotech Corporation). Using each DNA molecule as a template, PCR amplifications were performed using high-fidelity enzyme GVP8 (General Biosystems (Anhui) Corporation Limited). The PCR products were electrophoresed and gel-recovered to obtain a band of about 600 bp. The recovered PCR products were recombined with a pCDNA3.1 vector containing a signal peptide and human IgG Fc sequence (SEQ ID NO:53) to construct a cell expression plasmid expressing the fusion protein (Anti-PD1-Fc) consisting of anti-PD-1 single-domain antibody and human IgG4 Fc. Anti-PD1-Fc cell expression plasmids were then extracted using an endotoxin-free Plasmid Maxi Preparation Kit (Biomiga) and mixed with transfection reagent PEI (Polysciences, Inc.) at a ratio of 1:3, the product of which, after standing for 30 minutes, was added to HEK293F cells and cultured in a 37° C., 5% CO₂ shaker incubator for 7 days. The supernatant was then collected after centrifugation. The supernatant was adjusted to pH7.0 and loaded onto a Protein A affinity chromatography column (Bestchrom (Shanghai) Biosciences Ltd). Elution was performed with 100% 0.1M Gly-HCl (pH3.0); the eluent was mixed with 10% 1M Tris-HCl (pH8.5). The 100% eluent was diluted so that it has an electrical conductivity of 4 ms/cm and adjusted to pH 5.5. After centrifugation (8000 rpm, 4° C., 10 min), the supernatant was adjusted to pH 5.0 and loaded onto a DSP chromatography column (Bestchrom (Shanghai) Biosciences Ltd). Elution was performed with a linear gradient of 0-60% eluent (20 mM NaAc, 0.5M NaCl, pH5.0) at a flow rate of 2 ml/min for 15 minutes. Purity was measured using SEC-HPLC-UV analysis. Detector: Agilent 1100 LC; detection wavelength: 214 nm; mobile phase: 150 mM pH7.0 PB+5% isopropanol; column: Superdex 200 Increase 5/150 GL; run time: 15 minutes; column temperature: 25° C. The results showed that the purity was greater than 95%.

### Embodiment 6

Evaluation of In Vitro Functions of Anti-PD1-Fc Fusion Proteins

6.1 Evaluation of Binding Ability of Anti-PD1-Fc Fusion Proteins to Human PD-1

hPD1-HSA was coated on plates at 200 ng/well overnight at 4° C. and blocked with 5% skim milk at room temperature for 1 hour. The Anti-PD1-Fc fusion proteins were gradient diluted with 1% BSA and incubated at 37° C. for 1 hour. After washing with PBST 3 times, 100 μl of HRP-Goat anti-Human IgG Fc (Novex) diluted 1.20000 was added to each well and incubated at room temperature for 1 hour. After washing with PBST 3 times, TMB substrate was added and incubated at 37° C. After incubation for 5 min for color development, the reaction was terminated by the addition of 1M sulfuric acid and 2D450 nm values were read. The results are shown in Table 5.

TABLE 5

| | | SEQ | EC50 |
|---|---|---|---|
| | Sample codes | ID No. | (nM) |
| anti-PD-1 | Anti-PD1-1A4-Fc | 22 | 0.058 |
| single-domain | Anti-PD1-1A9-Fc | 23 | 0.058 |
| antibodies not | Anti-PD1-1H1-Fc | 24 | 0.051 |
| humanized | Anti-PD1-1D3-Fc | 25 | 0.056 |
| | Anti-PD1-2C4-Fc | 26 | 0.067 |
| | Anti-PD1-2D9-Fc | 27 | 0.078 |
| anti-PD-1 | Anti-PD1-hu1A4V1-Fc | 58 | 0.063 |
| single-domain | Anti-PD1- hu 1A9V1-Fc | 59 | 0.058 |
| antibodies humanized, | Anti-PD1- hu 1H1V1-Fc | 60 | 0.055 |
| with V1 and V3 | Anti-PD1- hu 1D3V1-Fc | 61 | 0.061 |
| representing two | Anti-PD1- hu 2C4V1-Fc | 62 | 0.058 |
| different humanized | Anti-PD1- hu 2D9V1-Fc | 63 | 0.062 |
| sequences | Anti-PD1- hu 1A4V3-Fc | 64 | 0.048 |
| | Anti-PD1- hu 1A9V3-Fc | 65 | 0.058 |
| | Anti-PD1- hu 1H1V3-Fc | 66 | 0.060 |
| | Anti-PD1- hu 1D3V3-Fc | 67 | 0.058 |
| | Anti-PD1- hu 2C4V3-Fc | 68 | 0.055 |
| | Anti-PD1- hu 2D9V3-Fc | 69 | 0.058 |
| Positive control | Keytruda ® | | 0.181 |

Note:
Keytruda ® is a marketed anti-PD1 monoclonal antibody (Merck).

6.2 Evaluation of Blocking Effect of Anti-PD1-Fc Fusion Proteins on hPD-L1/PD-1 Interaction (Competitive ELISA)

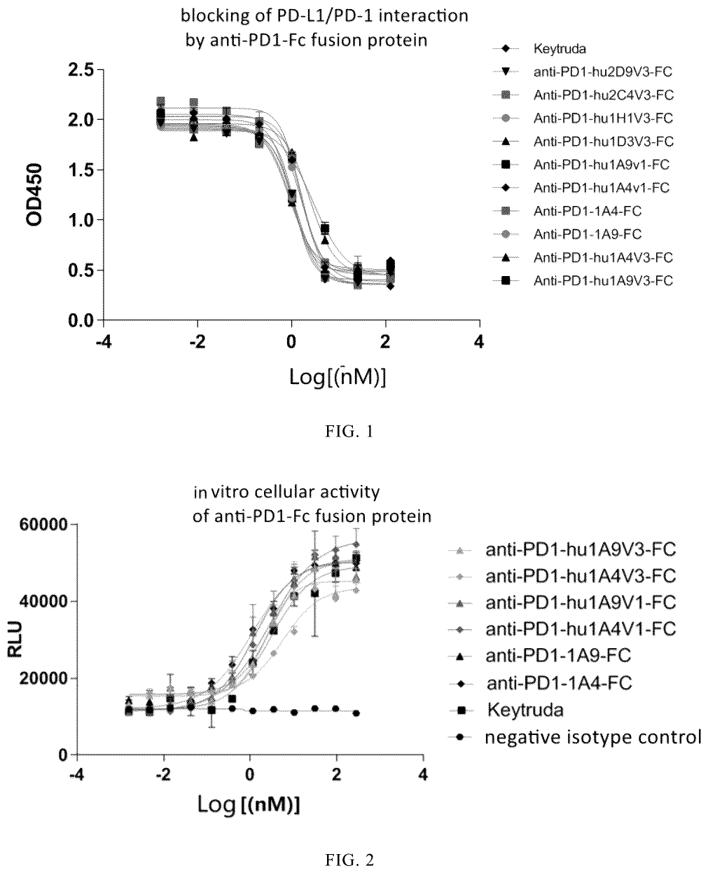

hPD1-HSA was coated on plates at 200 ng/well overnight at 4° C. and blocked with 5% skim milk at room temperature for 1 hour. The Anti-PD1-Fc fusion proteins was gradient diluted with 1% BSA from a starting concentration of 4 μg/mL in 5-fold increments to obtain a series of diluted solutions with decreasing concentrations, each of the diluted solutions was mixed with an equal volume of biotin-labeled 4 μg/mL bio-hPDL1-FC (SEQ ID NO.55), and 100 μL of each mixture was added to a 96-well plate and incubated at 37° C. for 1 hour. After washing with PBST 3 times, 100 μL of HRP-Streptavidin (Jackson ImmunoResearch Inc.) diluted 1:50000 was added to each well and incubated at room temperature for 1 hour. After washing with PBST 3 times, TMB substrate was added and incubated at 37° C. After incubation for 5 min for color development, the reaction was terminated by adding 1M sulfuric acid and OD450 nm values were read. The results are shown in Table 6 and FIG. 1.

TABLE 6

| | IC50 | | IC50 |
|---|---|---|---|
| Sample codes | (nM) | Sample names | (nM) |
| Anti-PD1-1A4-Fc | 1.454 | Anti-PD1-hu2C4V1-Fc | 1.212 |
| Anti-PD1-1A9-Fc | 1.554 | Anti-PD1-hu2D9V1-Fc | 1.032 |
| Anti-PD1-1H1-Fc | 1.535 | Anti-PD1-hu1A4V3-Fc | 2.43 |
| Anti-PD1-1D3-Fc | 1.486 | Anti-PD1-hu1A9V3-Fc | 2.842 |
| Anti-PD1-2C4-Fc | 1.549 | Anti-PD1-hu1H1V3-Fc | 1.121 |
| Anti-PD1-2D9-Fc | 1.437 | Anti-PD1-hu1D3V3-Fc | 0.8777 |
| Anti-PD1-hu1A4V1-Fc | 0.965 | Anti-PD1-hu2C4V3-Fc | 1.193 |
| Anti-PD1-hu1A9V1-Fc | 0.955 | Anti-PD1-hu2D9V3-Fc | 1.146 |
| Anti-PD1-hu1H1V1-Fc | 1.125 | Keytruda ® | 1.721 |
| Anti-PD1-hu1D3V1-Fc | 1.101 | | |

Note:
Keytruda ® is a marketed anti-PD1 monoclonal antibody (Merck).

The above results indicate that the Anti-PD1-Fc fusion proteins of the present disclosure is highly effective in blocking the hPDL-1/PD-1 interaction.

### Embodiment 7

Evaluation of Functional Activity of Anti-PD1-Fc Fusion Proteins in Human T Lymphocyte Cells

7.1 Construction of Assay Cell Lines

CD5L-OKT3scFv-CD14 (GenBank: ADN42857.1) was synthesized and digested with HindIII-EcoRI (Takara), then inserted into the pCDNA3.1 vector to construct pCDNA3.1-antiCD3TM. Using human PD-L1 (GenBank: NM_014143.2) as a template, high-fidelity amplification was performed to obtain PD-L1 fragments, which were then recombined with pCDNA3.1-antiCD3TM to construct pCDNA3.1-antiCD3TM-PDL1. CHO cells (Thermo) were transfected and then screened with G418 for 10-14 days to generate a stable cell line CHO-antiCD3TM-PDL1.

Using human PD1 (GenBank: NP_005009.2) as a template, fragments were obtained after amplification and recombined with the PB513B1-dual-puro vector (Youbio) digested with HindIII-BamHI (Takara) to construct the plasmid pB-PD1. Using pGL4.30 (Youbio) as a template, high-fidelity amplification was performed to obtain fragments, which were recovered and recombined with the pB-PD1 vector digested with SfiI-XbaI (Takara) to construct the plasmid pB-NFAT-Luc2p-PD1. The successfully constructed plasmids were extracted with the endotoxin-free Plasmid Maxi Preparation Kit (Biomiga) for transfection of Jurkat cells (Stem Cell Bank, Chinese Academy of Sciences). Following the method described in patent CN 107022571A, the Jurkat cells were treated with 0.1 mg/ml poly-D-lysine to become relatively adherent, and then transfected according to instructions of the liposome transfection kit (Lipofectamine 3000; Invitrogen). On the third day, selection under pressure was performed with RPM11640 medium (Thermo) containing 10% FBS and 2.5 μg/ml puromycin. Subsequently, medium was supplemented at intervals and puromycin concentration was gradually increased to 4 μg/ml after cell viability recovered. Finally, a monoclonal Jurkat-NFAT-Luc2p-PD1 cell line was obtained.

7.2 Functional Evaluation of Anti-PD1-FC Fusion Proteins Blocking hPD-L1/PD1 Pathway

CHO-CD3TM-PD-L1 and Jurkat-NFAT-Luc2p-PD-1 cells were counted and cell densities were adjusted to 4×10⁶/ml. 25 μl of each type of cells was added to each well in a 96-well plate. In this experiment, Anti-PD1-Fc fusion proteins, positive control Keytruda® (Merck), and negative isotype control were gradient diluted with 1% BSA respectively. 50 μl of the above antibodies were added to the cells to make the final concentration of the antibodies 280, 93.3, 31.1, 10.4, 3.5, 1.2, 0.4, and 0.1 nM. After co-culturing at 37° C. and 5% CO₂ for 6 hours, 10 μl of luciferase substrate (Promega, E2620) was added to each well, shaken for 2 minutes on an oscillator and read. The operation was performed according to instructions of the kit.

FIG. 2 shows that adding Anti-PD1-Fc fusion proteins block the binding of hPD-1/PD-L1 on surfaces of effector cells and target cells with characteristic dose-response curves indicating specificity, however, the negative isotype control cannot block the binding of hPD-L1 and PD1.

As can also be seen from FIG. 2, the ability of Anti-PD1-Fc fusion proteins, as optimized by the present disclosure, to block hPD-1/PD-L1 binding on the surfaces of the target cells was comparable to that of the positive control Keytruda®.

### Embodiment 8

Evaluation of Activation Effect of Anti-PD1-Fc Fusion Proteins on PBMC

5 μg/ml recombinant anti-CD3 monoclonal antibody (Novoprotein Scientific Inc, product number GMP-A018) was coated on plates at 50 μl/well overnight at 4° C. PBMC was isolated from the peripheral blood of healthy people using lymphocyte separation solution (Sigma) and diluted to 2×10⁶/ml with recombinant CD28 monoclonal antibody (Novoprotein Scientific Inc, product number GMP-A063) at a concentration of 2 μg/ml. The supernatant was discarded from the enzyme-labeled plates coated with recombinant anti-CD3 monoclonal antibody, and the plates were washed twice with 200 μl/well PBS, and 200 μl cells were added to each well. After cell activation for 24 hours, 5 nM Keytruda® and 5 nM Anti-PD1-Fc fusion protein were added for stimulation. After stimulation for 3 days, the supernatant was collected after centrifugation at 1500 rpm for 5 minutes and the supernatant was detected for cytokine IL2 secretion according to the instructions of the kit (purchased from Dakewe Biotech Co., Ltd.).

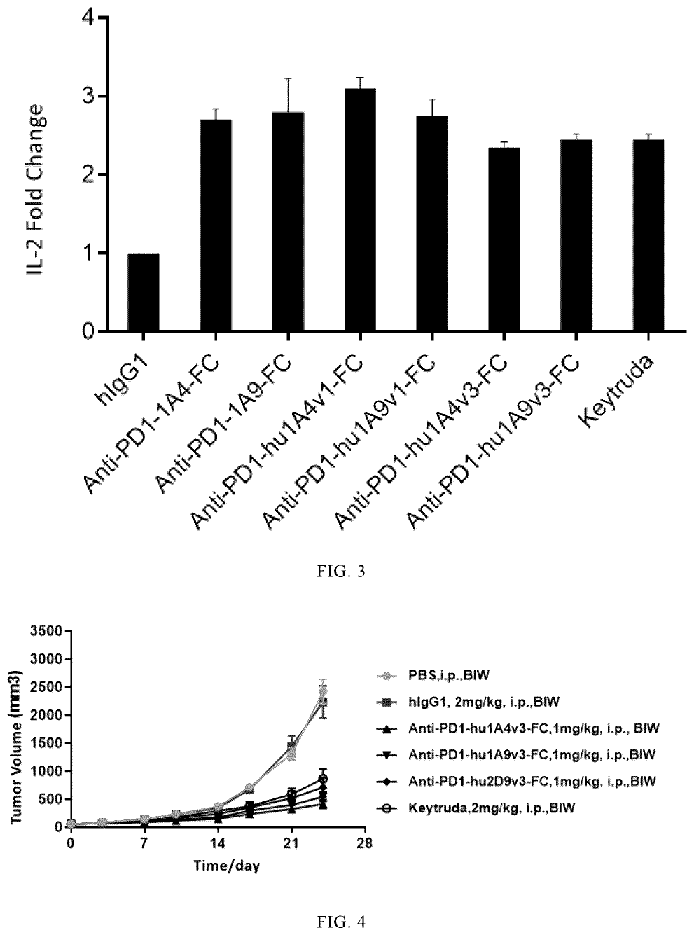

The results are shown in FIG. 3, Anti-PD1-Fc fusion proteins enhanced IL2 secretion by PBMC and the secretion of IL2 was significantly higher than that of positive control Keytruda®.

### Embodiment 9

Study of Tumor Inhibitory Activities of Anti-PD1-Fc Fusion Proteins

The tumor inhibitory activities of Anti-PD1-Fc fusion proteins was studied by establishing a colon cancer tumor model by subcutaneously transplanting MC38 cells expressing human PD-L1 (MC38-hPDL1) in humanized PD-1 C57 mice.

The experimental design was as follows: 1×10⁶ MC38-hPDL1 cells were subcutaneously inoculated into the right anterior leg of 6-8-week-old PD-1-humanized C57 mice. After inoculation, mice with tumor sizes of 50-70 mm³ were selected according to tumor volume and randomly divided into 6 groups, with 6 mice in each group. After grouping, Keytruda® (2 mg/kg), anti-PD1-hu1A4V3-Fc, anti-PD1-hu1A9V3-Fc, anti-PD1-hu2D9V3-Fc (each 1 mg/kg) and human IgG1 isotype control (2 mg/kg) were injected intraperitoneally respectively. The drugs were administered twice a week for a total of 8 times. PBS was injected in a parallel group as a negative control. Tumor volumes were measured twice a week.

Tumor volume measurement: the maximum length (L) and maximum width (W) of the tumor were measured using a vernier caliper and the tumor volume was calculated using the following formula: V=L×V²/2.

As shown in FIG. 4, as time went on, mice given anti-PD1-hu1A4V3-Fc, anti-PD1-hu1A9V3-Fc, and anti-PD1-hu2D9V3-Fc had their tumor volumes well controlled relative to the control group and did not show significant increases, indicating that Anti-PD1-Fc fusion proteins had significant tumor inhibitory effects.

### Embodiment 10

Study of Solubility of Anti-PD1-Fc Fusion Proteins

Purified 100 mg single-domain antibodies were ultrafiltered using ultrafiltration tubes (Merck Millipore Ltd.) to display their buffers with 5 mM phosphate buffer (pH7.2) or 5 mM sodium acetate (pH5.5). Ultrafiltration was performed at 25° C. and 3500 g and buffer displaced twice until further concentrated is not possible (meaning no significant change in volume after centrifugation for 20 minutes). After centrifugation at 8000 g for 10 minutes, protein concentrations of the concentrated solution were measured, which were then used to calculate the solubility of the anti-PD1-Fc fusion proteins under this condition.

TABLE 7

| Codes | SEQ ID No. | Solubilities(mg/ml) |
|---|---|---|
| Anti-PD1-hu1A4V3-Fc | 64 | 154.1 |
| Anti-PD1-hu1A9V3-Fc | 65 | 204.6 |
| Anti-PD1-hu1H1V3-Fc | 66 | 187.2 |
| Anti-PD1-hu1D3V3-Fc | 67 | 173.6 |
| Anti-PD1-hu2C4V3-Fc | 68 | 227.8 |
| Anti-PD1-hu2D9V3-Fc | 69 | 100.4 |

According to Table 7, the humanized Anti-PD1-Fc fusion protein as optimized by the present disclosure show good solubilities. The anti-PD-1 single-domain antibodies of the present disclosure was humanized by replacing the hydrophilic amino acids at positions 44 and 45 of the original FR2 region with the relatively conserved hydrophobic residues G and L of a normal human antibody (Table 4a), but this did not affect their solubilities.

The present disclosure effectively overcomes various shortcomings and a has high industrial value.

The above-mentioned embodiments are just used for exemplarily describing the principle and effects of the present disclosure instead of limiting the present disclosure. Those skilled in the art can make modifications or changes to the above-mentioned embodiments without going against the spirit and the range of the present disclosure. Therefore, all equivalent modifications or changes made by those who have common knowledge in the art without departing from the spirit and technical concept disclosed by the present disclosure shall be still covered by the claims of the present disclosure.

1. An anti-PD-1 single-domain antibody, which includes complementary determining regions including CDR1 having an amino acid sequence as set forth in one of SEQ ID Nos. 5 to 6, CDR2 having an amino acid sequence as set forth in one of SEQ ID Nos. 9 to 12, and CDR3 having an amino acid sequence as set forth in one of SEQ ID Nos. 17 to 21.

2. The anti-PD-1 single-domain antibody as in claim 1, wherein the complementary determining regions of the anti-PD-1 single-domain antibody includes CDR1 to CDR3 whose amino acid sequences are as follows:
(1) CDR1 has an amino acid sequence as set forth in SEQ ID No. 5, CDR2 has an amino acid sequence as set forth in SEQ ID No. 9, and CDR3 has an amino acid sequence as set forth in SEQ ID No. 17; or
(2) CDR1 has an amino acid sequence as set forth in SEQ ID No. 5, CDR2 has an amino acid sequence as set forth in SEQ ID No. 10, and CDR3 has an amino acid sequence as set forth in SEQ ID No. 18; or
(3) CDR1 has an amino acid sequence as set forth in SEQ ID No. 6, CDR2 has an amino acid sequence as set forth in SEQ ID No. 11, and CDR3 has an amino acid sequence as set forth in SEQ ID No. 19; or
(4) CDR1 has an amino acid sequence as set forth in SEQ ID No. 6, CDR2 has an amino acid sequence as set forth in SEQ ID No. 10, and CDR3 has an amino acid sequence as set forth in SEQ ID No. 18; or
(5) CDR1 has an amino acid sequence as set forth in SEQ ID No. 6, CDR2 has an amino acid sequence as set forth in SEQ ID No. 12, and CDR3 has an amino acid sequence as set forth in SEQ ID No. 20; or
(6) CDR1 has an amino acid sequence as set forth in SEQ ID No. 6, CDR2 has an amino acid sequence as set forth in SEQ ID No. 9, and CDR3 has an amino acid sequence as set forth in SEQ ID No. 21.

3. The anti-PD-1 single-domain antibody as in claim 1, wherein the anti-PD-1 single-domain antibody further includes framework regions FR, the framework regions FR include FR1 having an amino acid sequence as set forth in one of SEQ ID Nos. 1 to 3, FR2 having an amino acid sequence as set forth in SEQ ID No. 7, FR3 having an amino acid sequence as set forth in one of SEQ ID Nos. 13 to 15 and SEQ ID Nos. 28 to 29, and FR4 having an amino acid sequence as set forth in one of SEQ ID Nos. 31 to 32.

4. The anti-PD-1 single-domain antibody as in claim 3, wherein the framework regions FR include FR1 to FR4 whose amino acid sequences are as follows:
(1′) FR1 has an amino acid sequence as set forth in SEQ ID No. 1, FR2 has an amino acid sequence as set forth in SEQ ID No. 7, FR3 has an amino acid sequence as set forth in amino acid sequence SEQ ID No. 13, and FR4 has an amino acid sequence as set forth in SEQ ID No. 31; or
(2′) FR1 has an amino acid sequence as set forth in SEQ ID No. 1, FR2 has an amino acid sequence as set forth in SEQ ID No. 7, FR3 has an amino acid sequence as set forth in amino acid sequence SEQ ID No. 14, and FR4 has an amino acid sequence as set forth in SEQ ID No. 32; or
(3′) FR1 has an amino acid sequence as set forth in SEQ ID No. 1, FR2 has an amino acid sequence as set forth in SEQ ID No. 7, FR3 has an amino acid sequence as set forth in amino acid sequence SEQ ID No. 15, and FR4 has an amino acid sequence as set forth in SEQ ID No. 31; or
(4′) FR1 has an amino acid sequence as set forth in SEQ ID No. 1, FR2 has an amino acid sequence as set forth in SEQ ID No. 7, FR3 has an amino acid sequence as set forth in amino acid sequence SEQ ID No. 28, and FR4 has an amino acid sequence as set forth in SEQ ID No. 31; or
(5′) FR1 has an amino acid sequence as set forth in SEQ ID No. 2, FR2 has an amino acid sequence as set forth in SEQ ID No. 7, FR3 has an amino acid sequence as set forth in amino acid sequence SEQ ID No. 28, and FR4 has an amino acid sequence as set forth in SEQ ID No. 31; or
(6′) FR1 has an amino acid sequence as set forth in SEQ ID No. 3, FR2 has an amino acid sequence as set forth in SEQ ID No. 7, FR3 has an amino acid sequence as set forth in amino acid sequence SEQ ID No. 29, and FR4 has an amino acid sequence as set forth in SEQ ID No. 31.

5. The anti-PD-1 single-domain antibody as in claim 3, wherein the anti-PD-1 single-domain antibody is selected from:
a) single-domain antibodies, each having an amino acid sequence including an amino acid sequence as set forth in one of SEQ ID Nos. 34 to 39; and
b) single-domain antibodies, each having an amino acid sequence with a sequence identity of over 80% with an amino acid sequence as set forth in one of SEQ ID Nos. 34 to 39 and having functions of the single-domain antibodies as defined in a).

6. The anti-PD-1 single-domain antibody as in claim 1, wherein the anti-PD-1 single-domain antibody is a humanized antibody;
and/or, the anti-PD-1 single-domain antibody is derived from Vicugna pacos.

7. The anti-PD-1 single-domain antibody as in claim 6, which further includes framework regions FR including FR1 to FR4, wherein FR1 has an amino acid sequence as set forth in SEQ ID No. 4, FR2 has an amino acid sequence as set forth in one of SEQ ID Nos. 7 to 8, FR3 has an amino acid sequence as set forth in SEQ ID No. 16, and FR4 has an amino acid sequence as set forth in SEQ ID No. 33.

8. The anti-PD-1 single-domain antibody as in claim 7, wherein the anti-PD-1 single-domain antibody is selected from:
c) single-domain antibodies, each having an amino acid sequence including an amino acid sequence as set forth in one of SEQ ID Nos. 40 to 51; and
d) single-domain antibodies, each having an amino acid sequence with a sequence identity of over 80% with an amino acid sequence as set forth in one of SEQ ID Nos. 40 to 51 and having functions of the single-domain antibodies as defined in c).

9. A fusion protein, including a first domain and a second domain, wherein the first domain includes an anti-PD-1 single-domain antibody as in claim 1, and the second domain includes a domain capable of prolonging an in vivo half-life of the fusion protein and/or a domain capable of binding to effector cells.

10. The fusion protein as in claim 9, wherein the second domain includes one or more of an immunoglobulin Fc region, serum albumin or its fragment, a domain binding to serum albumin, polyethylene glycol, and a polyethylene glycol-liposome complex, wherein the immunoglobulin Fc region is preferably a human immunoglobulin Fc region.

11. The fusion protein as in claim 10, wherein the human immunoglobulin Fc region includes mutations for eliminating, attenuating or enhancing Fc-mediated effector functions, wherein the effector functions include one or more of CDC activity, ADCC activity, and ADCP activity.

12. The fusion protein as in claim 10, wherein the immunoglobulin is one or more of IgG, IgA1, IgA2, IgD, IgE, and IgM, and the IgG is one or more of IgG1, IgG2, IgG3, and IgG4 isoforms.

13. The fusion protein as in claim 10, wherein the immunoglobulin Fc region has an amino acid sequence as set forth in one of SEQ ID Nos. 52 to 53, and SEQ ID Nos. 74 to 79.

14. The fusion protein as in claim 9, further including a linker peptide between the first domain and the second domain, wherein the linker peptide is preferably a flexible polypeptide chain including alanine, and/or serine, and/or glycine, wherein the linker peptide preferably has a length of 3 to 30 amino acids.

15. The fusion protein as in claim 9, wherein the fusion protein has an amino acid sequence as set forth in one of SEQ ID No. 58 to 69.

16. An isolated polynucleotide, encoding an anti-PD-1 single-domain antibody as in claim 1, or encoding a fusion protein, wherein the fusion protein includes a first domain and a second domain, wherein the first domain includes the anti-PD-1 single-domain antibody, and the second domain includes a domain capable of prolonging an in vivo half-life of the fusion protein and/or a domain capable of binding to effector cells.

17. A construct including the isolated polynucleotide as in claim 16.

18. An expression system for an antibody, including the construct as in claim 17 or incorporating an exogenous polynucleotide, wherein the exogenous polynucleotide encodes the anti-PD-1 single-domain antibody or the fusion protein.

19. A method for preparing an anti-PD-1 single-domain antibody, or a fusion protein, wherein the method includes: culturing the expression system for an antibody as in claim 18 under conditions suitable for expressing the antibody or the fusion protein, thereby expressing the antibody or the fusion protein; and purifying and isolating the antibody or the fusion protein, wherein the anti-PD-1 single-domain antibody includes complementary determining regions including CDR1 having an amino acid sequence as set forth in one of SEQ ID Nos. 5 to 6, CDR2 having an amino acid sequence as set forth in one of SEQ ID Nos. 9 to 12, and CDR3 having an amino acid sequence as set forth in one of SEQ ID Nos. 17 to 21, wherein the fusion protein includes a first domain and a second domain, wherein the first domain includes the anti-PD-1 single-domain antibody, and the second domain includes a domain capable of prolonging an in vivo half-life of the fusion protein and/or a domain capable of binding to effector cells.

20. Use of an anti-PD-1 single-domain antibody as in claim 1, or a fusion protein in the preparation of a drug for the treatment of tumors, wherein the fusion protein includes a first domain and a second domain, wherein the first domain includes the anti-PD-1 single-domain antibody, and the second domain includes a domain capable of prolonging an in vivo half-life of the fusion protein and/or a domain capable of binding to effector cells.

21. The use as in claim 20, wherein the cancers include one or more of lung cancer, melanoma, gastric cancer, ovarian cancer, colon cancer, liver cancer, kidney cancer, bladder cancer, breast cancer, classical Hodgkin's lymphoma, hematological malignancy, head and neck cancer, and nasopharyngeal cancer.

22. A pharmaceutical composition including an anti-PD-1 single-domain antibody as in claim 1, or a fusion protein, wherein the fusion protein includes a first domain and a second domain, wherein the first domain includes the anti-PD-1 single-domain antibody, and the second domain includes a domain capable of prolonging an in vivo half-life of the fusion protein and/or a domain capable of binding to effector cells.
